(19) United States
(12) Patent Application Publication
(10) Pub. No.: US 2024/0200069 A1
(43) Pub. Date: Jun. 20, 2024

(54) Synthetic Near-Threshold Translational Repressors

(71) Applicant: Arizona Board of Regents on behalf of Arizona State University, Scottsdale, AZ (US)

(72) Inventors: Alexander Green, Scottsdale, AZ (US); Yu Zhou, Tempe, AZ (US)

(21) Appl. No.: 18/403,466

(22) Filed: Jan. 3, 2024

Related U.S. Application Data
(62) Division of application No. 17/155,387, filed on Jan. 22, 2021, now Pat. No. 11,898,144.
Application No. 17/155,387 is a division of application No. 16/322,799, filed on Feb. 1, 2019, now Pat. No. 10,941,401.
(60) Provisional application No. 62/369,521, filed on Aug. 1, 2016.

(51) Int. Cl. C12N 15/113; C12N 15/67; H03K 19/20

(52) U.S. Cl. CPC C12N 15/113; C12N 15/67; H03K 19/20; C12N 2310/122; C12N 2310/531

(57) ABSTRACT

Provided herein are synthetic nucleic acid molecules and methods of using such synthetic nucleic acid molecules for strong repression of target gene expression. In particular, provided herein are methods for altering expression of a protein in a cell, where the method comprises introducing into a cell a protein coding sequence operably linked to a near-threshold translational repressor having first and second trigger recognition sequences that are fully or partially complementary to a repressing trigger RNA; and introducing into a cell the repressing trigger RNA.

### CROSS-REFERENCE TO RELATED APPLICATIONS

This application is a divisional application of U.S. application Ser. No. 17/155,387, filed Jan. 22, 2021, which is a divisional application of U.S. application Ser. No. 16/322,799, filed Feb. 1, 2019, which granted on Mar. 9, 2021 as U.S. Pat. No. 10,941,401, and which represents the national stage entry of PCT International Application No. PCT/US2017/044815, filed on Aug. 1, 2017, which claims the benefit of U.S. Provisional Application No. 62/369,521, filed Aug. 1, 2016, each of which are incorporated herein by reference in their entireties.

### STATEMENT REGARDING FEDERALLY SPONSORED RESEARCH

Not applicable.

### REFERENCE TO AN ELECTRONIC SEQUENCE LISTING

The contents of the electronic sequence listing (112624.01436.xml; Size: 238,729 bytes; and Date of Creation: Dec. 27, 2023) is herein incorporated by reference in its entirety.

### BACKGROUND

Synthetic biology is an emerging discipline that has great potential to respond to global pandemics. The increasing ability of synthetic biologists to repurpose and engineer natural biological components for practical applications has led to new opportunities for molecular diagnostics. Although recent advances have demonstrated the advantages of de novo design for riboregulators that increase gene expression in response to a trigger RNA, systems that demonstrate the inverse behavior, a decrease in gene expression upon triggering, have yet to be reported. Accordingly, there remains a need in the art for improved compositions, methods, and systems for strong repression of target gene expression.

### BRIEF SUMMARY

In a first aspect, provided herein is a synthetic nucleic acid molecule comprising an RNA-sensing switch RNA sequence, wherein the switch RNA sequence comprises a near-threshold hairpin structure comprising a loop-forming region comprising a ribosomal binding site (RBS) and a stem-forming region comprising a start codon, and first and second trigger recognition sequences located 5′ and 3′ to the hairpin structure, respectively; wherein the hairpin structure is configured to alternatively adopt a translationally active ON state or adopt a translationally inactive OFF state in the presence of a repressing trigger RNA, and wherein the second trigger recognition sequence does not encode an in-frame stop codon. The start codon can be located in a region of non-complementarity. The first trigger recognition sequence can have a length of 15 nucleotides. The second trigger recognition sequence can have a length of 12 nucleotides. The first and second trigger recognition sequences can be fully or partially complementary to a repressing trigger RNA.

In another aspect, provided herein is a method for altering expression of a protein, the method comprising providing a protein coding sequence operably linked to a near-threshold translational repressor having first and second trigger recognition sequences that are fully or partially complementary to a repressing trigger RNA; and providing the repressing trigger RNA, whereby expression of the protein is altered according to the level of repressing trigger RNA provided. Expression of two or more proteins can be altered by providing two or more protein coding sequences, each operably linked to a near-threshold translational repressor comprising first and second trigger recognition sequences that are fully or partially complementary to a single repressing trigger RNA. The fold-change in expression of the protein can be at least 25 fold. The fold-change in expression of the protein can be at least 50 fold.

In another aspect, provided herein is a method for altering expression of a protein in a cell, the method comprising introducing into a cell a protein coding sequence operably linked to a near-threshold translational repressor having first and second trigger recognition sequences that are fully or partially complementary to a repressing trigger RNA; and introducing into a cell the repressing trigger RNA, whereby expression of the protein is altered according to the level of repressing trigger RNA introduced to the cell. Expression of two or more proteins can be altered by introducing into the cell two or more proteins coding sequences, each operably linked to a near-threshold translational repressor comprising first and second trigger recognition sequences that are fully or partially complementary to a single repressing trigger RNA. The fold-change in expression of the protein can be at least 25 fold. The fold-change in expression of the protein can be at least 50 fold.

In another aspect, provided herein is a synthetic nucleic acid NAND logic circuit, wherein the NAND logic circuit comprises two or more near-threshold translational repressor (NeaTTR) hairpin modules comprising a loop-forming region comprising a ribosomal binding site (RBS) and a stem-forming region comprising a start codon, and a trigger recognition sequence located 5′ and 3′ to the hairpin structure, wherein the NeaTTR hairpin modules are separated by a linker domain. The two or more NeaTTR hairpin modules can be operably linked to a downstream reporter element. The reporter element can be GFP or lacZ. The linker domain can be 11 or 17 nucleotides. The NAND logic circuit can comprise three NeaTTR hairpin modules. The NAND logic circuit can comprise four or more NeaTTR hairpin modules.

In another aspect, provided herein is a synthetic nucleic acid NOR logic circuit, wherein the NOR logic circuit comprises at least one input RNA sensing hairpin module comprising an input RNA binding domain and a loop domain comprising first and second trigger RNA sequences; and a near-threshold translational repressor (NeaTTR) hairpin module comprising a loop-forming region comprising a ribosomal binding site (RBS) and a stem-forming region comprising a start codon, and first and second trigger recognition sequences located 5′ and 3′ to the hairpin structure, respectively; wherein the first and second trigger recognition sequences are complementary to the first and second trigger RNA sequences, respectively, of the input RNA sending hairpin module. The NeaTTR hairpin module can be operably linked to a reporter element. The reporter element can be GFP or lacZ. The NOR logic circuit can comprise two RNA sensing hairpin modules. The NOR logic circuit can comprise three RNA sensing hairpin modules. The NOR logic circuit can comprise four or more RNA sensing hairpin modules.

The foregoing and other advantages of the invention will appear from the following description. In the description, reference is made to the accompanying drawings, which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF DRAWINGS

The present invention will be better understood and features, aspects, and advantages other than those set forth above will become apparent when consideration is given to the following detailed description thereof. Such detailed description makes reference to the following drawings, wherein:

FIG. 1 is a schematic illustrating design of exemplary synthetic near-threshold translational repressors (NeaTTRs). The switch RNA (top) contains a weak RBS-bearing hairpin with sequence recognition sites on both sides to respond to the trigger or target RNA. Downstream gene expression is inactivated by the robust three-way junction structure formed between the sensor and its cognate input trigger. The bottom of the switch RNA hairpin features a weak 5-bp stem engineered to form transiently and breathe open at temperatures near 37° C. This unstable structure enables the switch RNA to allow translation of the downstream gene in the absence of the trigger RNA and to facilitate trigger RNA binding to deactivate expression.

FIG. 2 is a graph depicting ON/OFF GFP fluorescence levels obtained 5 hours (hr) after induction with IPTG for 48 NeaTTRs. Relative errors for the switch ON/OFF ratios were obtained by adding the relative errors of the switch ON and OFF state fluorescence measurements from three biological replicates in quadrature. Inset: ON/OFF GFP fluorescence measured for repressors of varying performance levels at different time points (3 hr, 4 hr, 5 hr) following induction.

FIG. 3 presents representative flow cytometry histograms of GFP fluorescence for three NeaTTRs with varying performance levels. Flow cytometry measurements were taken 5 hours after induction. OFF state (repressor with cognate trigger) is displayed on top of ON state (repressor with silent trigger, a control RNA with high secondary structure). Flow cytometry GFP fluorescence histograms for the three repressors showed an ON/OFF ratio of 126, 280, and 783, respectively.

FIGS. 4A-4D present data of an assessment of NeaTTR orthogonality. (a) Fold change for all trigger-switch combinations. Fold change was determined by taking the GFP output for a trigger-switch combination and dividing it by the GFP output from the switch with its cognate trigger. (b) Percent repression levels for all trigger-switch combinations. Percent repression was calculated by subtracting fluorescence output from the average non-cognate fluorescence of this switch and then dividing by the average value. (c) Library dynamic range in fold change and orthogonal library size. (d) Library dynamic range in cross-repression and orthogonal library size. The overall dynamic range corresponds to the minimum ON/OFF ratio (c) or maximum cross-repression (d) to expect in a network employing an orthogonal sub-library of switches of the specified size.

FIGS. 5A-5C are graphs demonstrating in vitro validation of NeaTTRs by plate reader. (a) GFP synthesis kinetics for NeaTTR N08 in presence of its cognate or silent trigger. (b) GFP synthesis kinetics for NeaTTR N09 in presence of its cognate or silent trigger. (c) ON/OFF GFP ratio at four time points (1 hr, 2 hr, 3 hr, 4 hr).

FIGS. 6A-6B are schematics illustrating NOT-AND (“NAND”) gates based on NeaTTRs. (a) Type I NAND gate design. Multiple repressor switches are aligned in upstream of the regulated gene in their active state. Complementary triggers serve as input RNAs that are required to block all active switches to turn off the gene expression. (b) Type II NAND gate design. It requires two partially complementary triggers to hybridize and give a functional trigger to switch off the gene expression.

FIG. 7 is a schematic illustrating a N-Input NOT-OR (“NOR”) Gate RNA. A single NeaTTR module is used to detect a cognate trigger RNA of sequence a-b. An array of input RNA sensing hairpins upstream of the NeaTTR module contain trigger RNA sequences sequestered within their loops. Upon binding of an input RNA, the hairpin sensing domain is unwound to free the trigger sequence. The trigger sequence then binds to the NeaTTR module through an intramolecular refolding mechanism and in turn deactivates gene expression.

FIGS. 8A-8C present in vivo measurements of multiple-input Type I NAND gates constructed from concatenated NeaTTR hairpin modules. (A) Truth tables obtained for six different two-input NAND gates constructed from different pairs of NeaTTR modules regulating GFP. (B) Truth tables obtained for four different three-input NAND gates constructed from different sets of NeaTTR modules regulating GFP. (C) Truth tables obtained for three different four-input NAND gates constructed from different sets of NeaTTR modules regulating GFP. Fold reduction of GFP fluorescence represents the fold decrease in GFP fluorescence obtained for the different input conditions compared to the NULL input case where no cognate inputs are expressed. All circuits show the desired NAND logic behavior with large reductions in GFP output only occurring in the logical FALSE case with all input RNAs expressed in the cell. GFP expression remains relatively high for the logical TRUE cases where at least one input RNA is not expressed. The geometric mean of GFP fluorescence was calculated via flow cytometry 6 hours after induction of RNA expression with IPTG.

FIG. 9 presents in vivo measurements of 29 one-input (aka 1-bit) NOR constructs. Fold-change GFP from geometric mean is measured after 5 hours of induction of input and switch RNA expression in the cell. The one-input (aka 1-bit) NOR constructs can be used to construct multi-bit NOR gates.

FIG. 10 presents in vivo measurements of a two-input (aka 2-bit) NOR gate. Fold reduction in GFP from the geometric mean is measured after induction of input and switch RNA expression in the cell. The two-input NOR gate RNA was constructed by combining sensor domains from two validated one-input (1-bit) NOR constructs.

FIG. 11 presents in vivo measurements of NeaTTRs designed to detect full length mRNAs conferring antibiotic resistance and the E. coli small RNA ryhB. The mRNAs cat and kanR confer chloramphenicol and kanamycin resistance, respectively. GFP fluorescence was measured 5 hours after induction of RNA expression using IPTG.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the appended claims.

### DETAILED DESCRIPTION

All publications, including but not limited to patents and patent applications, cited in this specification are herein incorporated by reference as though set forth in their entirety in the present application.

The compositions, methods, and systems provided herein are based at least in part on the inventors' discovery of near-threshold translational repressors (NeaTTRs), a class of synthetic translational repressors comprising an RNA-sensing switch RNA and a repressing trigger RNA. The switch RNA possesses an unstable hairpin structure that can adopt a translationally active ON state or can enter a less active closed state to facilitate binding of the trigger RNA. We have designed and tested a library of 48 different NeaTTRs in E. coli. A subset of 20 NeaTTRs provide ON/OFF expression modulation of at least 50-fold, which corresponds to at least 98% repression. In contrast, previously reported translational repressors have been limited to at most 90%. Through comprehensive investigations of a library of 16 high performance devices, the inventors identified a set of 15 NeaTTRs that provide at least 40-fold repression and exhibit limited crosstalk interactions. NeaTTRs are also active in cell-free systems, signaling their potential use in paper-based or in vitro diagnostic devices. Also described herein are new methods of integrating NeaTTR elements into extended information-processing RNAs and trigger RNA complexes to achieve complex, multi-input logic expressions that can be deployed in vivo, in vitro, or on paper. NeaTTRs can also be used for the detection of nearly arbitrary RNAs, including endogenous RNAs expressed by the cell or those associated with pathogens.

For purposes of convenience in the description, references to nucleic acid elements such as start codons, ribosomal binding site, 5′ UTR, stem-loop, etc., may refer to either the RNA form or to the DNA form (i.e., to a DNA molecule that provides a template for transcription of the RNA). Similarly, when reference is made to modifying an RNA (e.g., by inserting an element such as a cis-repressive sequence) into the RNA, it is to be understood that the modification is generally accomplished by engineering the appropriate modification to a DNA molecule that provides a template for transcription of the RNA.

Accordingly, in a first aspect, provided herein is a synthetic RNA regulator that exploits a very unstable RNA secondary structure to enable signal output in the device ON state by facilitating ribosomal access and low OFF state expression by facilitating binding of a cognate trigger RNA with nearly arbitrary sequence. Such synthetic RNA regulators are referred to herein as “near-threshold translational repressors” (NeaTTRs) or “near-threshold repressor riboregulators.” As used herein, the term “near-threshold” refers to a switch RNA that has a secondary structure that is very close to being either in the translationally active or translationally repressed state, which enables the switch RNA to respond strongly to trigger RNA interactions that perturb its original secondary structure by a small number of base pairs.

As used herein, the terms “synthetic” and “engineered” are used interchangeably and refer to a non-naturally occurring nucleic acid molecule that has been created or modified by the hand of man (e.g., using recombinant DNA technology) or is derived from such a molecule (e.g., by transcription, translation, etc.). A nucleic acid molecule may be similar in sequence to a naturally occurring nucleic acid but typically contains at least one artificially created insertion, deletion, inversion, or substitution relative to the sequence found in its naturally occurring counterpart. A cell that contains a synthetic or engineered nucleic acid is considered to be an engineered cell.

In preferred embodiments, a near-threshold translational repressor is a synthetic nucleic acid molecule comprising an RNA-sensing switch RNA sequence, where the switch RNA sequence comprises an unstable hairpin structure comprising a loop-forming region comprising a ribosomal binding site (RBS) and a stem-forming region comprising an initiation codon, and first and second trigger recognition sequences located 5′ and 3′ to the hairpin structure, respectively; wherein the hairpin structure is configured to alternatively adopt a translationally active ON state or adopt a translationally inactive OFF state in the presence of a repressing trigger RNA, and wherein the second trigger recognition sequence does not encode an in-frame stop codon. A “hairpin” or “hairpin structure” refers to an intramolecular stem-loop structure of a nucleic acid sequence at the chosen assay temperature mediated by hybridization of partially or fully complementary sequences at the 5′- and the 3′-end of the nucleic acid sequence. As used herein, the term “unstable hairpin” refers to an RNA stem-loop structure that is not thermodynamically or kinetically favored (or is only weakly thermodynamically or kinetically favored). Such structures can adopt other secondary structures, either transiently or for longer periods of time, which can include additional unpaired bases. In some cases, unstable structures are characterized in terms of the fraction of time that they spend in the hairpin stem-loop form versus an unpaired structure where translation can occur. For example, the stem-loop structure may be more favorable (and be present for ˜51% of the time), but the stem-loop can open transiently and, thus, transiently adopt a more unstable structure, at which point the ribosome can bind and begin translation. The fraction of time spent in the different states depends on the sequence and the temperature. As used herein, the terms “trigger” and “trigger recognition sequence” refer to a RNA molecule capable of binding to single-stranded regions immediately upstream and downstream of the switch RNA hairpin in order to form a structure that represses translational output.

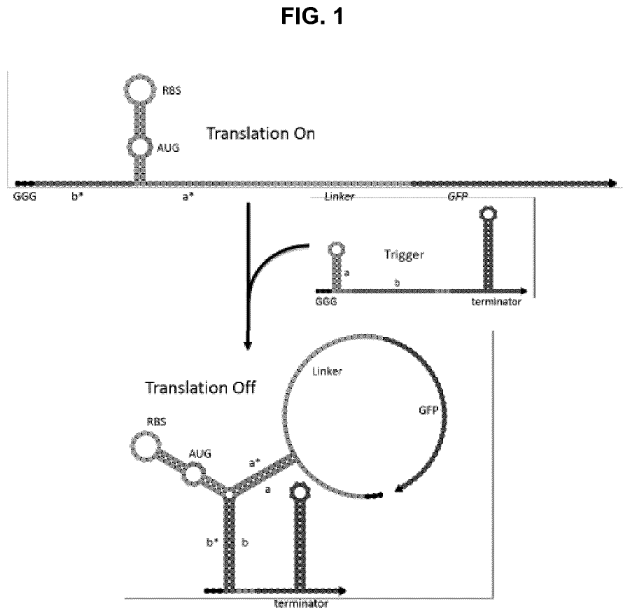

As described in the following paragraphs and the Example provided below, near-threshold translational repressors of the invention comprise generally switch and trigger RNAs designed to fully or partially hybridize with one another. The switch RNA contains the coding sequence of the target gene being regulated. Referring to FIG. 1, near-threshold translational repressors comprise a hairpin structure containing an RBS sequence (AGAGGAGA) within its loop is located upstream of the target gene's open reading frame. The hairpin structure is followed by stem-forming region comprising a start codon (AUG), situated 6 nucleotides (nts) downstream. Preferably, the hairpin structure comprises a stem having a total of 11 base pairs and a 3-nt region of non-complementarity (i.e., a bulge) formed by the start (initiation) codon. The upper part of this stem can comprise 6 base pairs and is sufficiently weak to enable access to the RBS by the ribosome. The bottom part of this stem is a weak 5-bp region with the sequence AACAA immediately downstream of the start codon. Importantly, this 5-bp region is sufficiently weak to make base pairing at the bottom of the hairpin transient. This unstable structure thus allows the ribosome to begin translation when the trigger RNA is absent. Upstream and downstream of the unstable hairpin are single-stranded trigger recognition sites that are 15 nts and 12 nts long, respectively. Between the 12-nt sensing sequence and the output gene is a linker sequence coding for a small additional peptides on the N terminus of the gene of interest. The loop of the switch can comprise 10 nts to 27 nts. The upper part of the stem can comprise 2 nts to 8 nts. The bottom part of the stem can comprise 2 nts to 8 nts. The trigger RNA comprises a 27-nt target sequence, where 10 nucleotides on its 5′ end are hidden within a small hairpin, leaving only 17 nts exposed to minimize unintended interactions between the switch and non-cognate triggers.

In some cases, the near-threshold translational repressors (NeaTTRs) described herein comprise trigger RNAs having toehold lengths of shorter than 17 nts. Without being bound by any particular theory or mechanism, it is expected that trigger RNAs can have shorter RNA toehold lengths, thus providing more substantial secondary structure, and retain the high repression levels of the riboregulators while having improved orthogonality. Accordingly, the NeaTTRs described herein include those having trigger types featuring fewer exposed single-stranded domains. In some cases, the toehold domain length of the trigger is decreased while the size of the stem in the trigger is increased for improved orthogonality. In other cases, the triggers do not feature any toehold but have a stem with a loop of 6 nts or more. Such loop-mediated triggers will first bind to the linear sensing site of the switch RNA and unwind their stems to bind through the full 27-nt recognition site.

In some cases, a NeaTTR as provided herein comprises a shortened trigger sequence that is predicted to provide strong repression. For example, triggers shorter than 10 nucleotides nucleotides (e.g., 10, 9, 8, 7, 6 nucleotides, inclusive) can be used to provide substantial repression and improved orthogonality. In addition, triggers that have regions shorter than 10 nucleotides (e.g., 10, 9, 8, 7, 6 nucleotides, inclusive) that bind to the switch RNA be used to provide substantial repression and improved orthogonality. In some cases, the shorter trigger sequences are used in addition to the 5-bp unstable stem. Without being bound to any particular mechanism or mode of action, it is understood that shorter triggers that reduce the post-start codon duplex length and bind to locations further to the 3′ end of the switch RNA will still provide substantial repression and offer improved orthogonality. Coupling these shortened triggers with new secondary structures yield further enhancement.

When the switch and cognate trigger RNAs are both present, the two recognition sequences flanking the RBS hairpin bind to the trigger RNA through a kinetically favorable linear-linear interaction between b and b* domains (FIG. 1). This linear-linear interaction is independent of the current folding state of the near-threshold switch hairpin. When the 5-bp bottom stem of the hairpin stem forms transiently, the two exposed bases of the trigger a domain are able to bind to the corresponding bases downstream of the hairpin. The stem of the trigger RNA then completes a branch migration process to unwind and enable complete binding of the trigger RNA to the switch. This interaction forms the final inactivated three-way junction structure. In both prokaryotic and eukaryotic systems, the ribosome must be able to gain access to the start codon. Since the inactivated three-way junction structure robustly sequesters the RBS and start codon, downstream protein translation is strongly repressed under such conditions.

It is important to note that the trigger RNA can also adopt a single-stranded structure and still repress the switch RNA. The hairpin structure in the trigger RNA depicted in FIG. 1 is utilized purely to reduce the number of exposed single-stranded bases in the trigger to decrease the likelihood of crosstalk between repressors when deployed in the same cell or reaction volume. In addition, other methods can be employed to reduce the number of exposed bases in the trigger, for instance by enclosing much of the binding sequence within the loop of a stem-loop structured trigger as described herein. Such a trigger RNA would exploit a loop-linear interaction in order to bind to and inactivate the switch RNA. The NeaTTR design places few constraints on the sequence of the trigger RNA. The only major constraint is the requirement that the recognition sequence downstream of the hairpin (domain a*) not code for an in-frame stop codon. This constraint can be avoided by moving the trigger RNA binding site by one or two nucleotides or by adding regions of non-complementarity (i.e., bulges) between the trigger RNA and the switch RNA to remove the stop codon.

As used herein, the term “non-complementarity” refers to refers to an entity in a double stranded region of an RNA composition (wherein the double strand nature of the RNA composition may arise from intramolecular hybridization within one RNA molecule and/or arise from intermolecular hybridization between two RNA molecules) that comprises non-complementary nucleotides between the two strands of the double stranded region. Thus, the region may be defined as a region of non-complementary nucleotides flanked by regions of double stranded RNA. In specific embodiments, the length of non-complementation is at least about 5 nucleotides. In other specific embodiments, the junction between the bubble and double stranded region comprises at least two T's. The terms “bubble” or “bulge” may also be used for the term “region of non-complementarity.” It will be understood that the terms “bubble” and “bulge” imply no specific shape of said region, although in some embodiments it is shaped as a bubble. Complementarity of two sequences is generally determined by dividing the total number of nucleotides that participate in complementary base pairs (GC, AU, AT) when the sequences are aligned to produce the maximum number of complementary base pairs, counting all nucleotides in the two sequences (including those in bulges, mismatches, or inner loops) by the total number of nucleotides contained in both sequences. For example, consider two sequences of 19 and 20 nucleotides in length in which alignment to produce the maximum number of complementary base pairs results in 16 base pairs, 1 inner loop of 2 nucleotides, 1 mismatch, and 1 bulge (in the sequence with 20 nucleotides). The percent complementarity of the two sequences is [(16+17)/39]100. It will be appreciated that complementarity may be determined with respect to the entire length of the two sequences or with respect to portions of the sequences. As used herein, two sequences are considered “substantially complementary” herein if their complementarity is at least 50%.

As shown in FIG. 1, near-threshold translational repressors are in some cases operably linked to a reporter element (e.g., an E. coli lacZ reporter element encoding ß-galactosidase) that is 3′ to the hairpin structure. As used herein, the term “operably linked” refers to a relationship between two nucleic acid sequences wherein the production or expression of one of the nucleic acid sequences is controlled by, regulated by, modulated by, etc., the other nucleic acid sequence. Reporter proteins appropriate for the methods provided herein include, without limitation, enzymatic reporters (e.g., β-galactosidase, alkaline phosphatase, DHFR, CAT), fluorescent or chemiluminescent reporters (e.g., GFP, GFP variants, mCherry, luciferase, e.g., luciferase derived from the firefly (Photinus pyralis) or the sea pansy (Renilla reniformis) and mutants thereof), etc.

In another aspect, provided herein are methods of altering gene expression using the synthetic nucleic acid molecules described herein. In some cases, the method alters expression of an enzyme (or other protein) in a cell and comprises introducing into a cell an enzyme coding sequence operably linked to a near-threshold translational repressor having first and second trigger recognition sequences that are fully or partially complementary to a repressing trigger RNA; and introducing into a cell the repressing trigger RNA, whereby expression of the enzyme is altered according to the level of repressing trigger RNA introduced to the cell.

In some cases, near-threshold translational repressors can be used to regulate expression of one or more polypeptides, including polypeptides representing entire metabolic pathways. In such cases, the method includes placing one or more polypeptides (e.g., an enzyme in a metabolic pathway) under control of NeaTTRs that respond to the same trigger RNA. The inventors have demonstrated that the near-threshold translational repressors provided herein yield unexpectedly high fold-change values, often in the 100-fold range. As used herein, the terms “expressing,” “expression,” or “express” refer to the production of a gene product (e.g., an mRNA transcript from a nucleic acid sequence encoding thereof). As used herein, the terms “gene product” and “expression product” generally refer to an RNA transcribed from the gene or a polypeptide encoded by an RNA transcribed from the gene. Thus a regulatory element, environmental condition, stimulus, etc., that alters the level of transcription or the stability of an RNA transcribed from a gene or alters its ability to serve as a template for translation will be said to alter expression of the gene. Similarly, a regulatory element, environmental condition, stimulus, etc., that alters the level of translation or stability of a polypeptide translated from an RNA transcribed from the gene will be said to alter expression of the gene.

Near-threshold translational repressors have multiple potential applications in vitro for diagnostic purposes. Integration of these near-threshold translational repressors into a low-cost, portable, and stable cell-free platform, such as one described by Pardee et al., Cell 159:940-954 (2014) and Pardee et al., Cell 165:1255-1266 (2016), could enable the applications below to be realized at under $1 per test and deployed either in the field, in remote clinics, at the point of care, or even in the home.

In some cases, near-threshold translational repressors are incorporated into complex, multi-input logic circuits. For diagnostic purposes, multi-input logic circuits can be used to increase assay specificity or sensitivity. For instance, a NOT-AND (“NAND”) expression can reduce false positives by ensuring that more than one pathogen-associated RNA is present in a sample. A NOT-OR (“NOR”) expression can reduce false negatives by sensing more than one RNA in the same sample. In vivo, a multi-input logic circuit can be used to sense the set of RNAs produced by a prokaryote in response to environment stresses. This RNA signature can be used to modulate production of enzymes or identify cells that are susceptible to antibiotics or screen for new antibiotic compounds. In addition, the circuits that sense a set of RNAs expressed by the host could be used to generate whole-cell biosensors that detect toxic chemicals in the environment.

A first synthetic nucleic acid logic circuit is the NOT-AND (“NAND”) gate RNA. As described herein, one type of NAND gate RNAs has a Type 1 geometry and comprise two or more NeaTTR hairpin modules. The two or more NeaTTR hairpin modules are separated by a linker sequence. In some embodiments, the linker sequence is 11-nt or 17-nt in length such that the NeaTTR hairpins remain in the same reading frame and to ensure that they are single stranded when expressed as part of the gate RNA. In some embodiments the two or more NeaTTR hairpin modules are upstream of an operably linked reporter element. In some embodiments, the reporter element is GFP or lacZ. In some embodiments, the NAND gate comprise two NeaTTR hairpin modules. In some embodiments, the NAND gate comprises three NeaTTR hairpin sequences. In some embodiments the NAND gate comprises four NeaTTR hairpin sequences. In some embodiments, the NAND gate comprises more than four NeaTTR hairpin sequences. It is envisioned that each NeaTTR module in a single NAND gate RNA maybe be specific to a separate input RNA trigger sequences.

As described herein, a second type of NAND gate RNA has a Type 2 geometry and comprises a NeaTTR hairpin module, wherein the NeaTTR hairpin module comprises two input RNA recognition sites that are partially complementary to a first and a second input RNA sequence. The two input RNA recognition sites are located on the 5′ and 3′ regions upstream and downstream of the hairpin and each domain is designed to complement a separate input RNA sequence. For example, the 5′ upstream input RNA recognition sequence is complementary to a first input RNA sequence and the 3′ downstream input RNA recognition sequence is complementary to a second input RNA sequence. The two input RNAs are also partially complementary to one another and form a two-RNA complex that efficiently binds to the two input RNA recognition sites to repress translation from the NeaTTR hairpin module. In some embodiments, the input RNA complex comprises three partially complementary input RNAs that binds to the NeaTTR hairpin module. In some embodiments, the input RNA complex comprises four partially complementary input RNAs that binds to the NeaTTR hairpin module. In some embodiments, the input RNA complex comprises more than four partially complementary input RNAs that binds to the NeaTTR hairpin module.

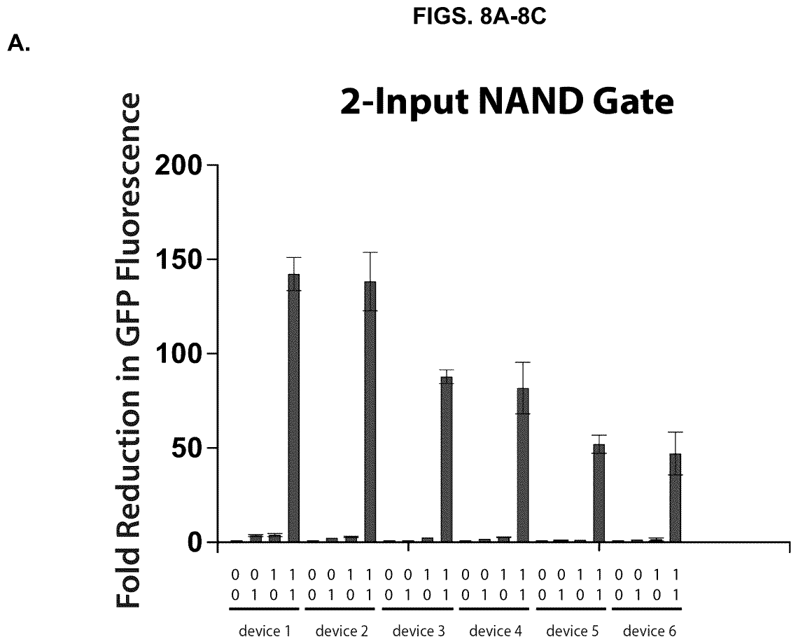

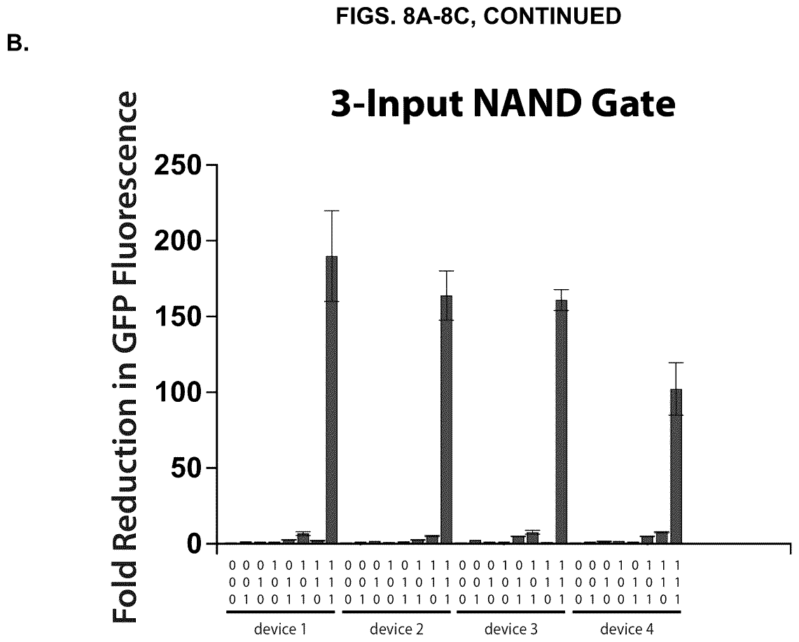

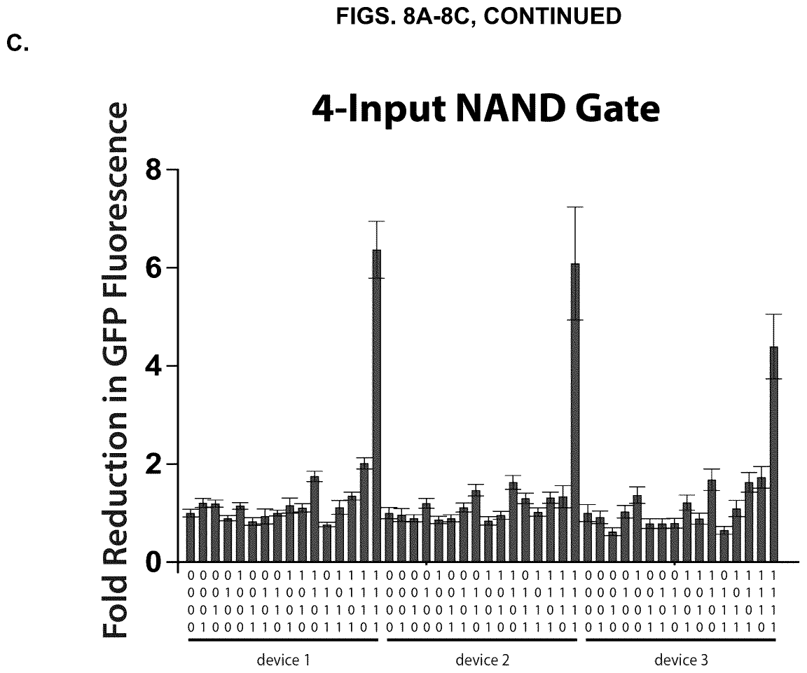

As described in the Example that follows, multiple NAND gate RNAs have been successfully implemented using validated NeaTRRs in Type I NAND geometries. For example, NAND gate RNAs were constructed by concatenating multiple NeaTTR hairpin modules upstream of the GFP open reading frame. The hairpin modules were separated by 11-nt or 17-nt spacer RNA sequences. These spacers ensured that the NeaTTR hairpins remained in the same reading frame and were designed to ensure that they were single stranded when expressed as part of the gate RNA. Referring now to FIG. 8, flow cytometry can be used to measure the geometric mean GFP fluorescence of cells expressing NAND gates and different combinations of input RNAs. The fold reduction in GFP was calculated by taking the GFP fluorescence measured for cells not expressing any cognate input RNAs (i.e., the NULL input case) and dividing it by the GFP fluorescence measured for cells expressing a specific combination of input RNAs. Measurements of six different two-input NAND gate circuits are shown in FIG. 8A. The NAND gate RNAs were constructed from pairs of NeaTTR hairpins upstream of GFP. For all devices, high fold reductions in GFP were obtained only when both input RNAs are expressed, which corresponds to the sole logical FALSE condition. In the other input combinations, GFP levels were not substantially reduced, as expected for these logical TRUE conditions. All six two-input NAND circuits thus show the desired NAND logical behavior. FIG. 8B presents measurements from a set of four different three-input NAND circuits. These circuits employed three NeaTTR hairpin modules concatenated upstream of GFP. Strong reductions in GFP output were only observed for the logical FALSE condition with all input RNAs expressed, while much smaller changes are observed for the logical TRUE states. FIG. 8C shows measurements from three exemplary four-input NAND circuits constructed from combinations of four NeaTTR hairpin modules arrayed upstream of GFP. These circuits also show the desired GFP output behavior in response to different combinations of input RNAs. The four-input devices showed lower fold reductions in general compared to the two-input and three-input circuits; however, the observed reductions were still substantially higher for the logical FALSE state compared to the logical TRUE states.

A second synthetic nucleic acid logic circuit is the NOT-OR (“NOR”) gate RNA. As described herein, the NOR gate RNA comprises one or more RNA sensing hairpin modules comprising trigger RNA sequences within a loop domain and an input RNA binding domain, wherein the one or more RNA sensing hairpin modules are upstream of a NeaTTR module comprising an unstable hairpin structure comprising a loop-forming region comprising a ribosomal binding site (RBS) and a stem-forming region comprising an initiation codon, and first and second trigger recognition sequences located 5′ and 3′ to the hairpin structure, respectively. The trigger sequences within the loop domain of the RNA sending hairpin modules are complementary to trigger recognition sequences within the NeaTTR module. Binding of an input RNA to the input binding domain of the RNA sensing hairpin module initiates an intramolecular refolding reaction in which the trigger sequences of the RNA sensing hairpin bind the complementary trigger recognition sequences of the NeaTTR. In some embodiments, the NeaTTR is operably linked to a downstream reporter sequence. In some embodiments, the reporter sequence is a sequence encoding GFP or lacZ. In some embodiments, one input RNA sensing hairpin module is upstream of the NeaTTR module. In some embodiments, two input RNA sensing hairpin modules are upstream of the NeaTTR module. In some embodiments, three or more RNA sensing hairpin modules are upstream of the NeaTTR module. It is envisioned that each RNA sensing hairpin module in a single NOR gate RNA maybe be specific to a separate input RNA sequence.

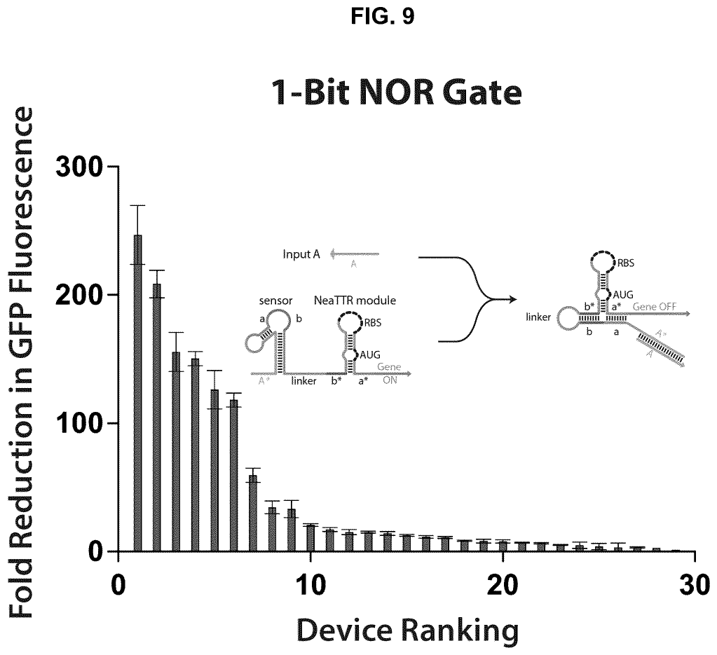

With respect to “NOR” gates, FIG. 9 illustrates the basic molecular mechanism of NOR gate RNAs in a simplified 1-bit system in which a single sensor hairpin is positioned upstream of a NeaTTR module. In such cases, a trigger RNA A can bind to the A* binding site in the sensor hairpin and unwind its stem. This event then exposes an intramolecular trigger containing the a and b sequences that can then bind to the downstream NeaTTR module at sites a* and b* to repress translation of the output gene. As described in the Example that follows, flow cytometry can be used to measure gate RNAs several hours after induction of RNA expression using IPTG. The gate RNAs displayed wide variations in function, with some displaying very little change in GFP output upon expression of the cognate input RNA. However, 7 out of the 29 devices (24%) provided at least 50-fold reductions in GFP expression upon detection of the input RNA and two provided very large reductions of over 200-fold.

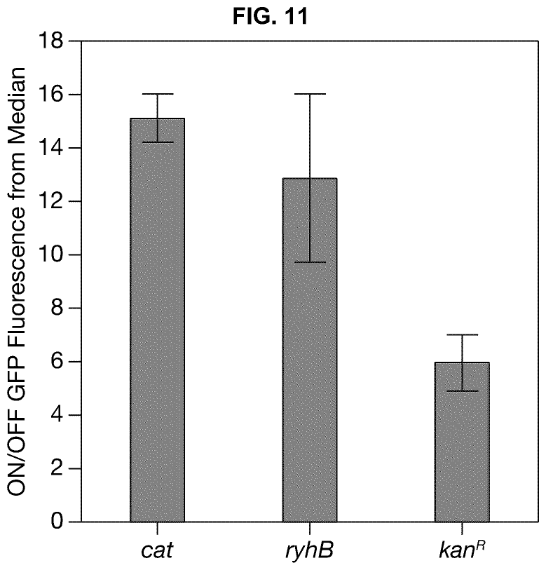

In some cases, near-threshold translational repressors are useful in diagnostic methods. Devices that repress translation can be used for turning off expression of an enzyme that is otherwise fed forward in the circuit (e.g. a transcription factor or an RNA polymerase). This type of control can halt all downstream reactions based on the downstream gene. In some cases, near-threshold translational repressors are useful for the detection of natural RNAs. Since the a* and b* domains within NeaTTR hairpins (referring to FIG. 1) can adopt virtually any sequence, they can be used to detect natural RNAs associated with drug resistance, viruses, hereditary diseases, and microorganisms. Referring now to FIG. 11, NeaTTRs can be responsive to natural RNAs. For example, systems can be configured to turn off expression of GFP in response to detection of their target RNAs and bind to the target sequence 5′-b-a-3′. In some cases, reductions of GFP fluorescence are 6-fold or more. Referring now to FIG. 11, an exemplary sensor, which was configured to detect the catalase mRNA (cat) responsible for chloramphenicol resistance, provided a fold reduction of 15 upon cat binding. Riboregulators that turn off protein expression in response to a target pathogen-, resistance- or disease-related RNA could be used, for instance, as kill switches that trigger cell death when a bacterium picks up a drug resistance mutation or a resistance-conferring plasmid (e.g., a toxin-antitoxin system can be disrupted when the riboregulator ceases production of the antitoxin). These riboregulators can also be deployed using cell-free systems for RNA detection in a diagnostic device.

NeaTTRs can also be used to implement time-dependent reactions and/or control the amount of a protein produced. For example, a switch RNA can be transcribed at the outset of the diagnostic reaction to begin production of its output protein. At a later time, a trigger RNA can be added or expressed that turns of production of the output protein, thus creating a time-dependent expression profile. Repressors can also be used in complementary reactions with activating riboregulators. In this context, a repressor would be used to detect the same target RNA as an activator. Interaction of the target RNA with both riboregulators would result in changes in the production of two enzymes, and yield clearer diagnostic results. Repressor systems can also be used in diagnostic systems for control reactions to determine if a technician is carrying out an assay properly. For instance, the repressor can be used to detect a control RNA or DNA that is amplified along with a sample from a patient. If repressor output is low, it means that the amplification reaction was successful (provided the cell-free reaction is active). If repressor output is high, it means that the amplification reaction failed but that the cell-free diagnostic stage was successful. An activating riboregulator to detect a control RNA would also be used for such a set of validation assays to determine if the cell-free reaction is functional when amplification is successful.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

The present invention has been described in terms of one or more preferred embodiments, and it should be appreciated that many equivalents, alternatives, variations, and modifications, aside from those expressly stated, are possible and within the scope of the invention. The invention will be more fully understood upon consideration of the following non-limiting Examples.

### Example

This example demonstrates near-threshold translational repressors (NeaTTRs) and uses thereof.

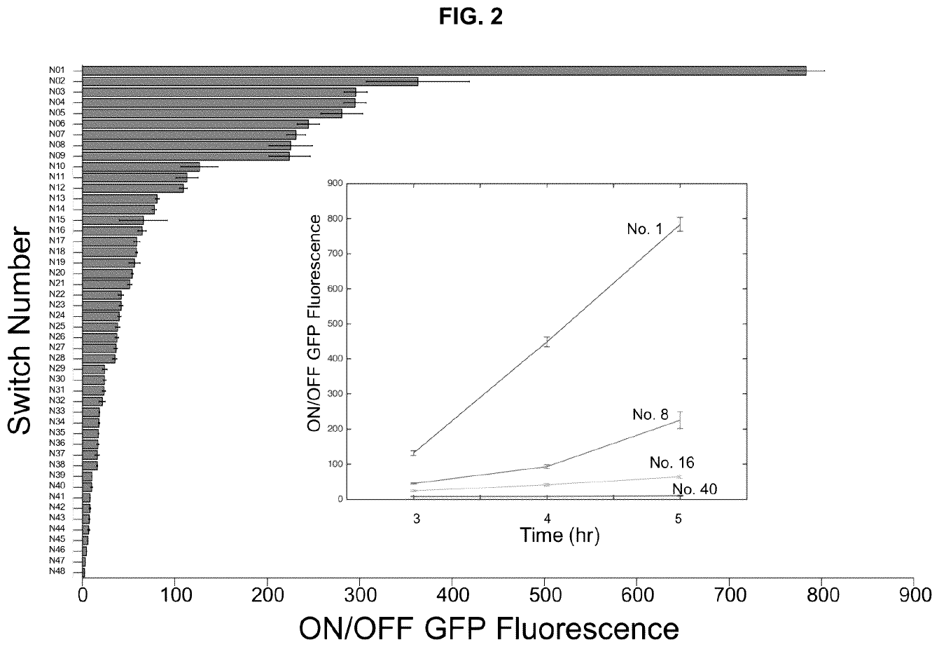

In Vivo NeaTTR Validation: A total of 48 NeaTTRs were designed and constructed (Table 1), and the 48 pairwise interactions between the cognate trigger RNA and a non-cognate trigger RNA with high secondary structure (the “silent” trigger) were tested in E. coli BL21 star DE3. Green fluorescent protein (GFP) was used as the output protein for characterizing switch output through flow cytometry. ON state expression levels were obtained from cells expressing the switch and a non-cognate trigger. OFF state expression levels were obtained from cells expressing the switch and its cognate trigger. Flow cytometry histograms were then used to obtain the median GFP expression from both ON and OFF strains and then used to compute the ON/OFF ratio. Considerable variations in ON/OFF ratios among all repressors were observed (FIG. 2), but overall the cognate pairs showed significant repression with an average of 50-fold change in GFP output. Out of the 48 devices, 12 provided ON/OFF levels exceeding 100-fold and 5 provided ON/OFF exceeding 250-fold. Thus a substantial fraction of these NeaTTRs provided high dynamic range without any additional parameter tuning from empirical measurements. We expect that further improvements can be gained through forward engineering approaches we have previously described⁶.

Time-course measurements revealed increasing levels of repression over time as ON state cells produced increasing amounts of GFP. A set of four different NeaTTRs with four different levels of dynamic range are shown in the inset of FIG. 2. Differences in the ON/OFF levels of the four devices increased over time to a final value of approximately 780-fold for the highest performance NeaTTR (NeaTTR No. 1) after 5 hours of induction.

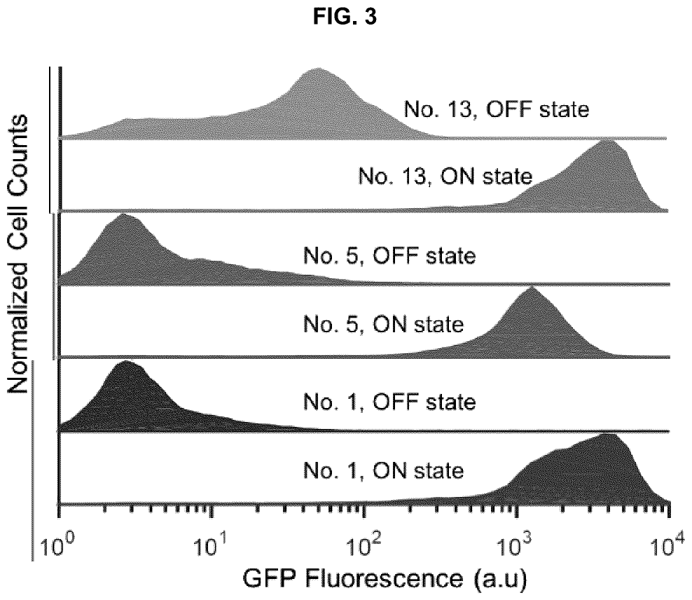

Representative flow cytometry histograms of three different repressors are provided in FIG. 3. In general, the OFF state of the repressors display wider fluorescence distributions. In particular, NeaTTR 13 possessed a nearly bimodal OFF state distribution with the dominant population at the higher fluorescence level. This distribution lead to the overall lower ON/OFF ratio obtained for NeaTTR 13 compared to devices 1 and 5. The ON state expression of the devices exhibited much tighter distributions overall and provide very high absolute levels of translation.

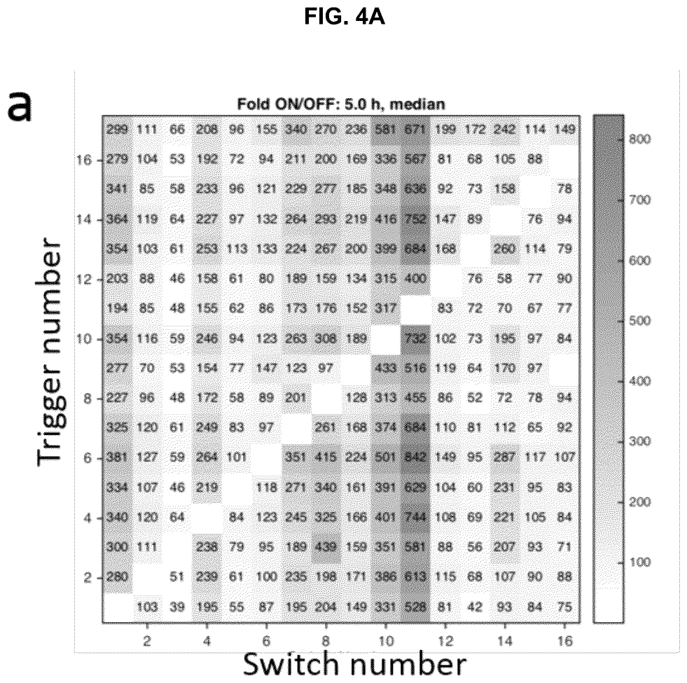

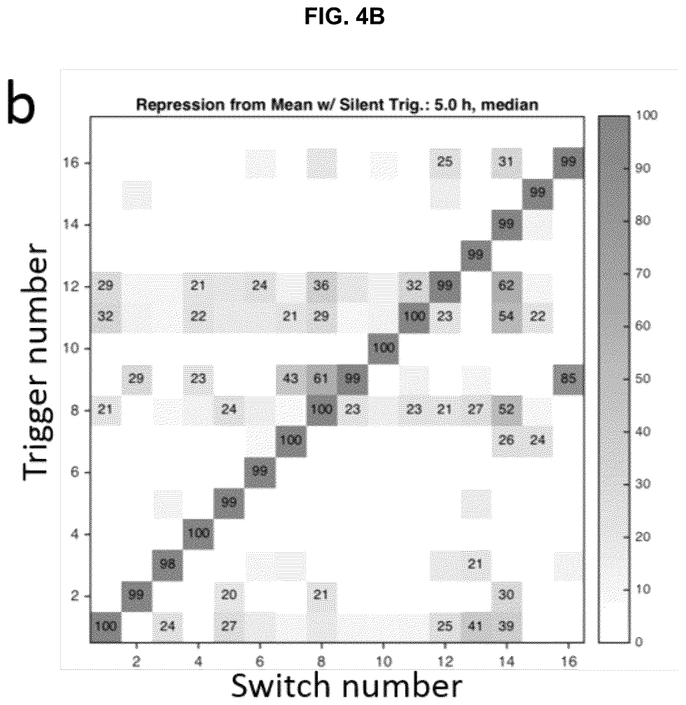

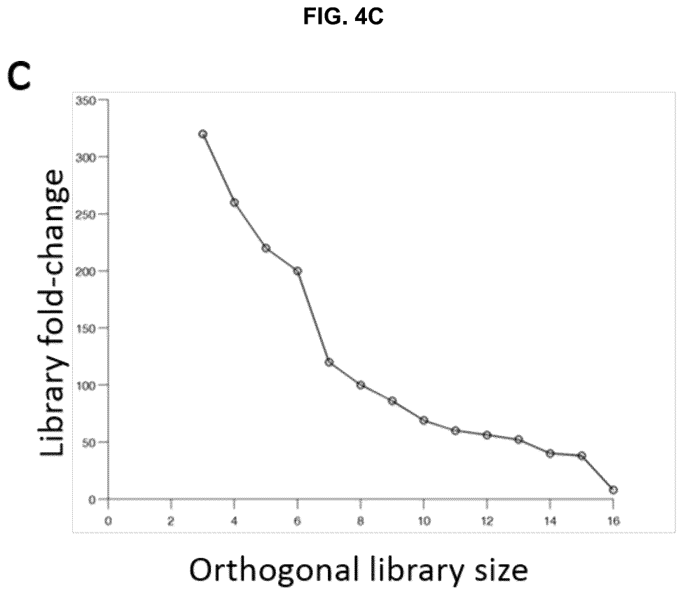

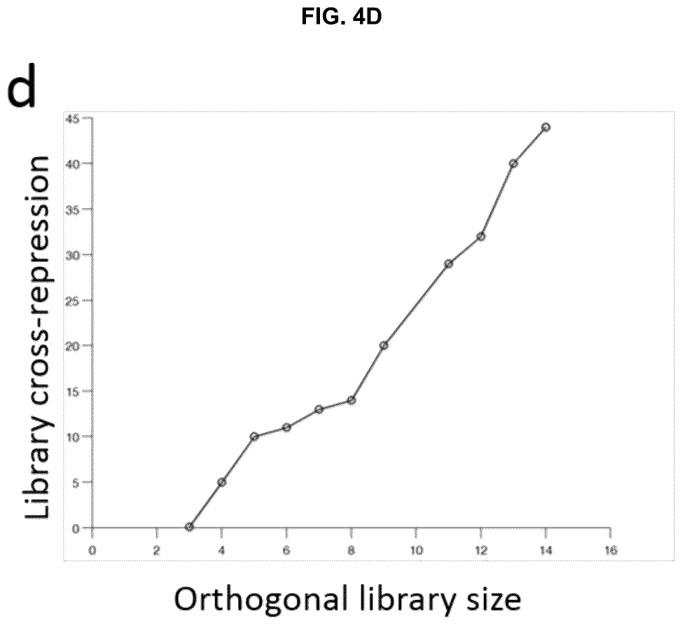

Evaluation of NeaTTR orthogonality: To measure the orthogonality of the devices, we isolated a subset of 16 repressors that displayed high ON/OFF GFP levels at 5 hours and assayed in E. coli all 256 pairwise combinations of switch and trigger plasmids. The silent trigger with high secondary structure was also tested as the 17th trigger for all switches. We used flow cytometry to quantify GFP output from all interactions. FIGS. 4A-4B provide orthogonality matrices depicting the degree of crosstalk between devices. FIG. 4a shows the ON/OFF levels obtained from all pairwise interactions. These values were obtained along each column by taking the fluorescence level for each interaction with the switch RNA and dividing it by the fluorescence level obtained with cognate trigger RNA. Thus, all values along the diagonal of FIG. 4a are equal to 1. Superimposed on most of the elements in the orthogonality matrix are numbers giving the ON/OFF level for each pairwise combination having a fold change of at least 40. Almost all noncognate pairs showed over 50-fold GFP fluorescence output compared with its cognate pair. FIG. 4b provides an orthogonality matrix obtained from percent repression levels. These percent repression levels were determined by first calculating the average GFP expression level of the switch RNA when expressed with each of the 15 non-cognate triggers in the library and the silent trigger. This mean ON state expression level was then used to determine the percent repression levels for all 17 different triggers expressed against a given switch. Based on this definition using the mean ON state, roughly half of the switch-trigger combinations will actually provide enhanced expression levels and negative percent repression. Although this metric may sound counter-intuitive, it does provide a good measure of the typical percent repression to expect for a switch RNA when the cognate trigger is expressed. Furthermore, it removes the effects of outlier expression levels obtained for specific non-cognate triggers that can exaggerate the degree of crosstalk between devices.

Using the orthogonality matrices shown in FIGS. 4a-4b, we then determined subsets of the 16 member high performance NeaTTR library that provided a desired threshold level of crosstalk. In terms of fold-change measurements, 15 riboregulators formed a subset expected to provide at least 40-fold ON/OFF levels if used in combination (FIG. 4c). This 40-fold mark means that you would expect to have a 40-fold difference between each repressor in the orthogonal set when its corresponding trigger RNA is expressed compared to the ON-state expression level when any of the other non-cognate triggers is expressed. For more stringent crosstalk requirements, a subset of 8 devices will provide at least 100-fold dynamic range and a subset of 6 devices 200-fold dynamic range. FIG. 4d depicts the size of orthogonal subsets obtained when applying the same considerations to the percent repression experimental data. An orthogonal subset of 8 devices, for instance, will provide cross-repression levels of below 15% if deployed at the same time. A subset of 9 devices will provide cross-repression levels of at most 20%. Previously reported repressing riboregulators have demonstrated lower degrees of orthogonality compared to the ones demonstrate here. Mutalik et al. showed that they could generate a set of six translational riboregulators with under 20% crosstalk⁴, while Takahashi described a library of seven transcriptional attenuators with under 20% crosstalk in percent repression. Using shortened triggers or those with reduced trigger sequence accessibility, we expect that the orthogonality of our repressors can be substantially improved. Furthermore, our repressors demonstrate markedly higher fold-change values often in the 100-fold range while those of Mutalik et al. and Takahashi et al. are generally below 20-fold.

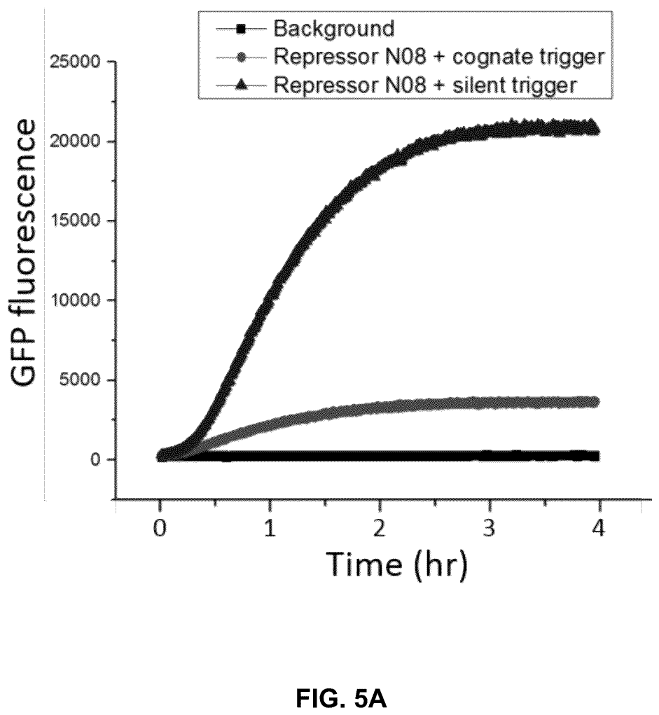

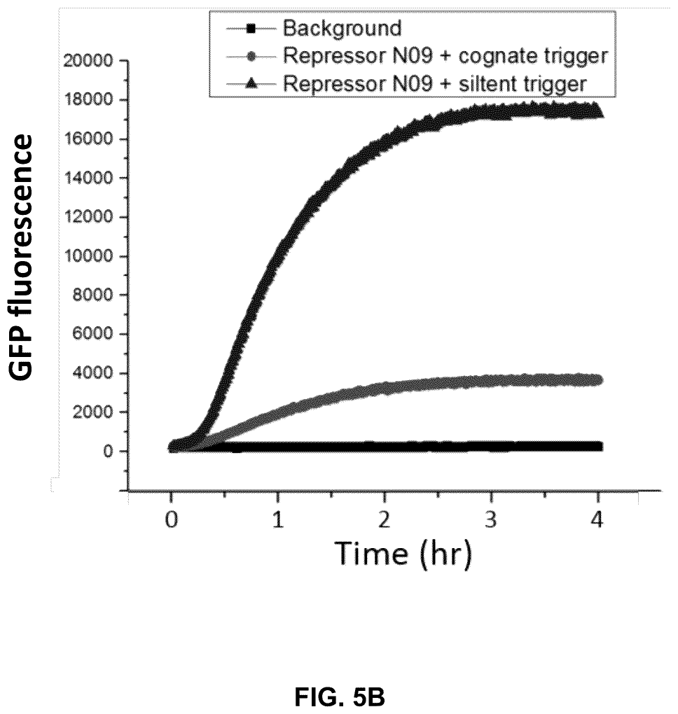

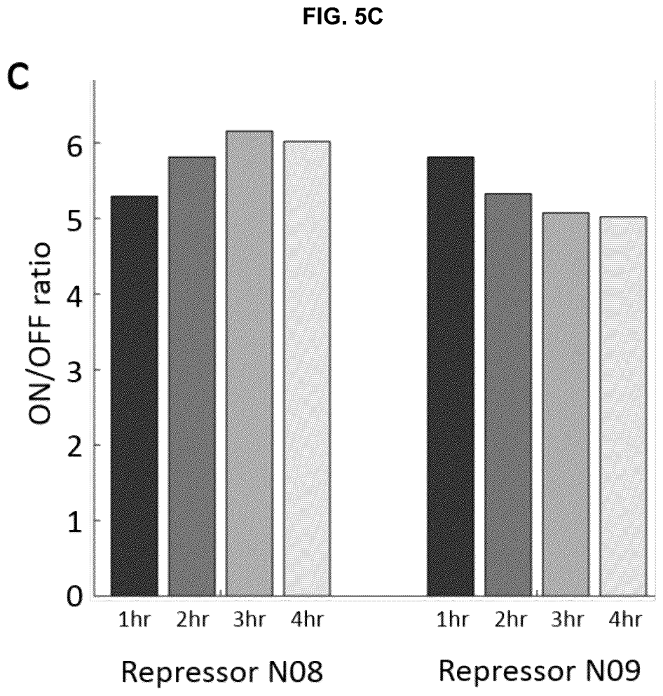

In vitro validation by plate reader: Cell-free tests were conducted to validate NeaTTR performance in vitro. Plasmids for transcribing two representative switches were mixed with their cognate trigger or silent trigger in 1:4 ratio in cell free reaction system, and GFP synthesis kinetics was monitored by plate reader as shown in FIGS. 5a-5b. A cell-free reaction system without any plasmids was used as background which was later subtracted from GFP output for calculating GFP ON/OFF ratio in FIG. 5c. Switches with cognate triggers clearly showed significant repression in GFP synthesis, and displayed 5 to 6 fold ON/OFF GFP ratio after 1 hour of reaction. The successful use of NeaTTRs in cell-free systems demonstrates that these riboregulators could be used for regulating synthetic gene networks in vitro and could be deployed as part of cell-free diagnostics used either in liquid form or in paper-based (or, more generally, porous substrate) reaction systems^{10,11}.

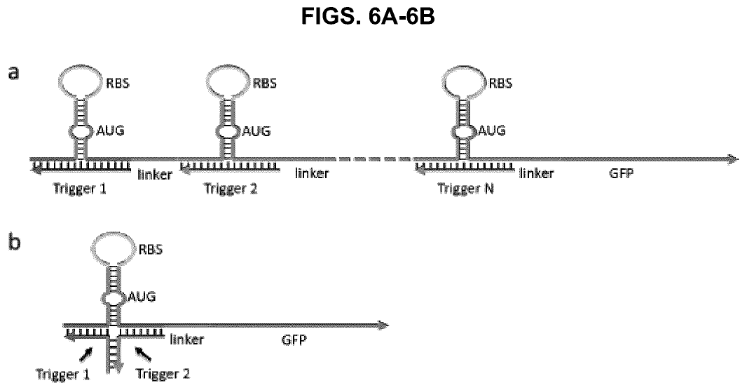

In vivo evaluation of NeaTTR Type I NAND logic gates: Tests of three Type I NAND gates (FIG. 6A) were conducted in E. coli. Each of these NAND gates consists of two NeaTTR unstable switches upstream of a GFP reading frame and thus evaluates the expression NAND(A,B). Input A corresponds to the trigger RNA that binds to the 5′ NeaTTR switch module and input B corresponds to the trigger RNA that binds to the 3′ NeaTTR switch module.

FIGS. 8A-8C show the performance of multiple NAND gate RNAs hours after induction of RNA transcription. We have successfully implemented multiple NAND gate RNAs using validated NeaTTRs in Type I NAND geometries. NAND gate RNAs were constructed by concatenating multiple NeaTTR hairpin modules upstream of the GFP open reading frame. The hairpin modules were separated by 11-nt or 17-nt spacer RNA sequences. These spacers ensured that the NeaTTR hairpins remained in the same reading frame and were designed to ensure that they were single stranded when expressed as part of the gate RNA. Data were obtained by using flow cytometry to measure the geometric mean GFP fluorescence of cells expressing the NAND gates and different combinations of input RNAs. The fold reduction in GFP was calculated by taking the GFP fluorescence measured for cells not expressing any cognate input RNAs (i.e., the NULL input case) and dividing it by the GFP fluorescence measured for cells expressing a specific combination of input RNAs. Measurements of six different two-input NAND gate circuits are shown in FIG. 8A. See Table 4. The NAND gate RNAs were constructed from pairs of NeaTTR hairpins upstream of GFP. For all devices, high fold reductions in GFP are obtained only when both input RNAs are expressed, which corresponds to the sole logical FALSE condition. In the other input combinations, GFP levels are not substantially reduced, as expected for these logical TRUE conditions. All six two-input NAND circuits thus show the desired NAND logical behavior.

FIG. 8B presents measurements from a set of four different three-input NAND circuits (Table 5). These circuits employed three NeaTTR hairpin modules concatenated upstream of GFP. Strong reductions in GFP output were only observed for the logical FALSE condition with all input RNAs expressed, while much smaller changes were observed for the logical TRUE states. FIG. 8C shows measurements from three different four-input NAND circuits (Table 6) constructed from combinations of four NeaTTR hairpin modules arrayed upstream of GFP. These circuits also showed the desired GFP output behavior in response to different combinations of input RNAs. The four-input devices showed lower fold reductions in general compared to the two-input and three-input circuits; however, the observed reductions were still substantially higher for the logical FALSE state compared to the logical TRUE states.

### Validation of “1-Bit” NOR Gates

We have confirmed the basic molecular mechanism of the NOR gate RNAs using a simplified 1-bit system in which a single sensor hairpin is positioned upstream of a NeaTTR module (FIG. 9, inset). In this gate RNA, a trigger RNA A can bind to the A* binding site in the sensor hairpin and unwind its stem. This event then exposes an intramolecular trigger containing the a and b sequences that can then bind to the downstream NeaTTR module at sites a* and b* to repress translation of the output gene.

A library of 29 different 1-bit NOR gates (Table 2) was constructed based on the same NeaTTR module and tested for regulation of GFP as the output protein. The gate RNAs were measured via flow cytometry five hours after induction of RNA expression using IPTG. The gate RNAs displayed wide variations in function, with some displaying very little change in GFP output upon expression of the cognate input RNA. However, 7 out of the 29 devices (24%) provided at least 50-fold reductions in GFP expression upon detection of the input RNA and two provided very large reductions of over 200-fold.

### Validation of a Two-Input NOR Gate

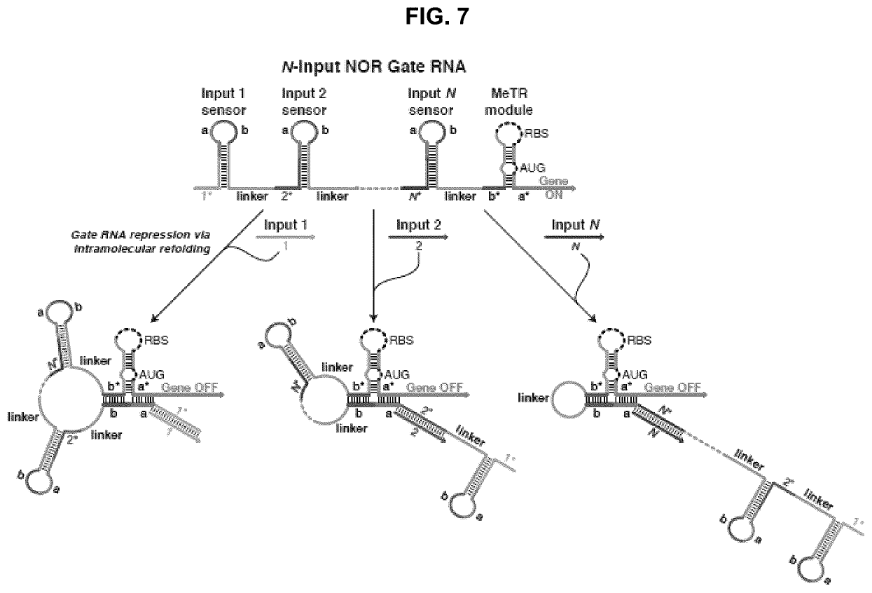

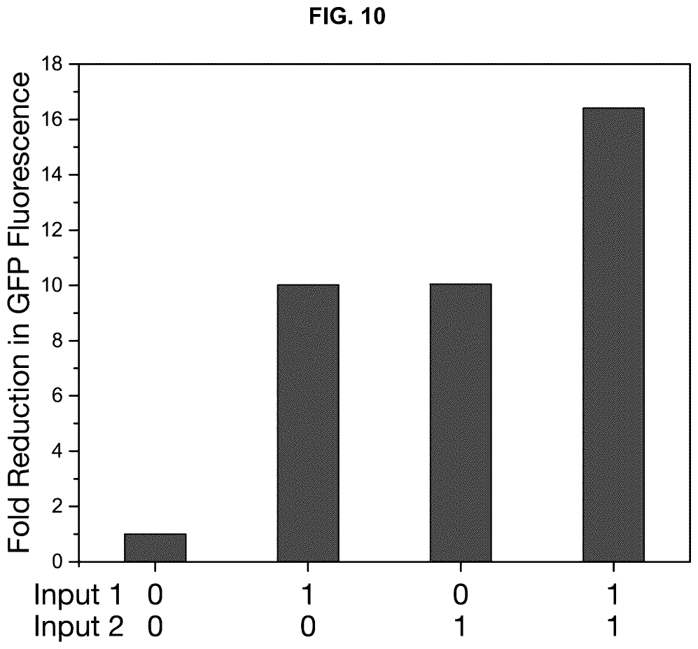

We have successfully implemented a two-input NOR gate based on the general design depicted in FIG. 7. The system features a NeaTTR domain downstream of two hairpins that detect different input RNAs (see Table 2) and harbor intramolecular triggers within their loops. The two-input NOR gate was constructed using the previously validated 1-bit NOR constructs, specifically the 2^{nd} and 4^{th} ranked devices shown in FIG. 9. The NOR gate was measured five hours after induction of circuit RNA expression using IPTG. FIG. 10 shows the fold reduction in GFP expression for the four possible combinations of input RNAs 1 and 2. The highest GFP expression levels are obtained when neither of the inputs is expressed. In the presence of any of the input RNAs, GFP fold reductions of at least 10 are observed, as expected for a functional NOR gate.

### Detection of Natural RNAs Using NeaTTRs

Since the a* and b* domains within NeaTTR hairpins (FIG. 1) can adopt virtually any sequence, they can be used to detect natural RNAs associated with drug resistance, viruses, hereditary diseases, and microorganisms. FIG. 11 displays results obtained from measurements in E. coli of three different NeaTTRs responsive to natural RNAs. These systems turn off expression of GFP in response to detection of their target RNAs and bind to the target sequence 5′-b-a-3′. We observed reductions of GFP fluorescence of 6-fold or more. The best performing sensor, which detects the catalase mRNA (cat) responsible for chloramphenicol resistance, provided a fold reduction of 15 upon cat binding. Riboregulators that turn off protein expression in response to a target pathogen-, resistance- or disease-related RNA could be used, for instance, as kill switches that trigger cell death when a bacterium picks up a drug resistance mutation or a resistance-conferring plasmid (e.g., a toxin-antitoxin system can be disrupted when the riboregulator ceases production of the antitoxin). These riboregulators can also be deployed using cell-free systems for RNA detection in a diagnostic device.

All three gates show at least 10-fold increases in GFP expression within 3 hours of induction compared to the sole logical FALSE case with inputs A and B present. Fold-change values increase with induction time for all three NAND gates. The circuit shown in FIG. 8B provides the most even GFP expression levels for the three logical TRUE cases, with fold changes of between 40 and 50 at the 5-hour time point. The circuit shown in FIG. 8C yields the highest fold-change levels overall with between 60- to 152-fold for the 5-hour time point. The success of these three NeaTTR-based gate RNAs demonstrates the robustness of the Type I design for constructing multi-input NAND logic.

TABLE 1
NeaTTR Devices

| Device # | Device name | Switch RNA Sequence | Trigger RNA sequence |
|---|---|---|---|
| 1 | STUR_NN_gen | GGGAUGAAUGAUAUACACUUGUUA | GGGACGAAUUGA |
| | 1_N020 | UAGUUAUGAACAGAGGAGACAUAA | UUUGUCAAUUCGU |
| | | CAUGAACAAGCACGAAUUGACUAC | GCGUGUAUAUCAU |
| | | ACUAAACCUGGCGGCAGCGCAA | UCAUCAU (SEQ ID |
| | | (SEQ ID NO: 1) | NO: 101) |
| 2 | STUR_NN_gen | GGGUAGUAAGAUUAGAUAUUGUUA | GGGACAAGAACAA |
| | 1_N010 | UAGUUAUGAACAGAGGAGACAUAA | UACGGUUCUUGUA |
| | | CAUGAACAAGUACAAGAACCGAAA | CUAUCUAAUCUUA |
| | | CGAGAACCUGGCGGCAGCGCAA | CUAAAC (SEQ |
| | | (SEQ ID NO: 2) | ID NO: 102) |
| 3 | STUR_WW_gen | GGGUACUCAAGAAUUCCAUUGUUA | GGGACAUACUAUA |
| | 1_N008 | UAGUUAUGAACAGAGGAGACAUAA | UCGUUAGUAUGU |
| | | CAUGAACAAUCACAUACUAACAUU | GAUGGAAUUCUU |
| | | ACUAAACCUGGCGGCAGCGCAA | GAGUAAUG (SEQ |
| | | (SEQ ID NO: 3) | ID NO: 103) |
| 4 | STUR_NN_gen | GGGACUACUAUUCACUUAUUGUUA | GGGCGACACUAAU |
| | 1_N021 | UAGUUAUGAACAGAGGAGACAUAA | AGUGUAGUGUCG |
| | | CAUGAACAAUCCGACACUACAAUU | GAUAAGUGAAUA |
| | | UCGAAACCUGGCGGCAGCGCAA | GUAGUAGA (SEQ |
| | | (SEQ ID NO: 4) | ID NO: 104) |
| 5 | STUR_NN_gen | GGGACAAUCAAAUACAAAUUGUUA | GGGACAUACAAUG |
| | 1_N013 | UAGUUAUGAACAGAGGAGACAUAA | AACUUUGUAUGU |
| | | CAUGAACAAUCACAUACAAAGCAA | GAUUUGUAUUUG |
| | | ACGAAACCUGGCGGCAGCGCAA | AUUGUAGC (SEQ |
| | | (SEQ ID NO: 5) | ID NO: 105) |
| 6 | STUR_NN_gen | GGGAUGAUAUUGAAUUACUUGUUA | GGGACACUACAAC |
| | 1_N002 | UAGUUAUGAACAGAGGAGACAUAA | UCAGUGUAGUGU |
| | | CAUGAACAAGCACACUACACUAAA | GCGUAAUUCAAUA |
| | | CAUAAACCUGGCGGCAGCGCAA | UCAUACU (SEQ |
| | | (SEQ ID NO: 6) | ID NO: 106) |
| 7 | STUR_NN_gen | GGGUCCAAAUCUUACUUAUUGUUA | GGGACUACCUCAC |
| | 1_N015 | UAGUUAUGAACAGAGGAGACAUAA | ACUCGAGGUAGUG |
| | | CAUGAACAAUCACUACCUCGAAUA | AUAAGUAAGAUU |
| | | CUUAAACCUGGCGGCAGCGCAA | UGGAAGU (SEQ |
| | | (SEQ ID NO: 7) | ID NO: 107) |
| 8 | STUR_NN_gen | GGGCAAAUACUCCAUAUCUUGUUA | GGGACAUAAACCU |
| | 1_N023 | UAGUUAUGAACAGAGGAGACAUAA | AUGAGUUUAUGU |
| | | CAUGAACAAGCACAUAAACUCACU | GCGAUAUGGAGU |
| | | GAUAAACCUGGCGGCAGCGCAA | AUUUGAAA (SEQ |
| | | (SEQ ID NO: 8) | ID NO: 108) |
| 9 | STUR_NN_gen | GGGCUCCUAUCACUUUACUUGUUA | GGGACACUAACCA |
| | 1_N024 | UAGUUAUGAACAGAGGAGACAUAA | UAUAGUUAGUGU |
| | | CAUGAACAAGCACACUAACUACAA | GCGUAAAGUGAU |
| | | AUUCAACCUGGCGGCAGCGCAA | AGGAGUAA (SEQ |
| | | (SEQ ID NO: 9) | ID NO: 109) |
| 10 | STUR_NN_gen | GGGAGUAUAAGAUAGAUAUUGUUA | GGGACGAAGCAUA |
| | 1_N003 | UAGUUAUGAACAGAGGAGACAUAA | AAGUUGCUUCGUA |
| | | CAUGAACAAGUACGAAGCAACGAA | CUAUCUAUCUUAU |
| | | CAUAAACCUGGCGGCAGCGCAA | ACUAAC (SEQ |
| | | (SEQ ID NO: 10) | ID NO: 110) |
| 11 | STUR_NN_gen | GGGACUAAUCAGAUCUACUUGUUA | GGGACCUAACAUA |
| | 1_N019 | UAGUUAUGAACAGAGGAGACAUAA | AACUUGUUAGGU |
| | | CAUGAACAAGCACCUAACAAGACU | GCGUAGAUCUGAU |
| | | AAUCAACCUGGCGGCAGCGCAA | UAGUGUG (SEQ |
| | | (SEQ ID NO: 11) | ID NO: 111) |
| 12 | STUR_NN_gen | GGGUGAUUGAAUAAGAAUUUGUUA | GGGCGAUAAUGU |
| | 1_N012 | UAGUUAUGAACAGAGGAGACAUAA | UCCGUCAUUAUCG |
| | | CAUGAACAAGCCGAUAAUGACACU | GCAUUCUUAUUCA |
| | | AACUAACCUGGCGGCAGCGCAA | AUCAUAG (SEQ |
| | | (SEQ ID NO: 12) | ID NO: 112) |
| 13 | STUR_WW_gen | GGGCAAAGUAUCCAUCAUUUGUUA | GGGACAUACUAAC |
| | 1_N010 | UAGUUAUGAACAGAGGAGACAUAA | AAUCUAGUAUGU |
| | | CAUGAACAAUCACAUACUAGAAUC | GAAUGAUGGAUA |
| | | UAAGAACCUGGCGGCAGCGCAA | CUUUGAAA (SEQ |
| | | (SEQ ID NO: 13) | ID NO: 113) |
| 14 | STUR_NN_gen | GGGCAAGAUUUAGUAGAUUUGUUA | GGGCGAACGACGA |
| | 1_N007 | UAGUUAUGAACAGAGGAGACAUAA | AACGGUCGUUCGG |
| | | CAUGAACAAUCCGAACGACCGAAA | AAUCUACUAAAUC |
| | | UGAUAACCUGGCGGCAGCGCAA | UUGAAA (SEQ |
| | | (SEQ ID NO: 14) | ID NO: 114) |
| 15 | STUR_NN_gen | GGGCAAUUAGUACUAUCCUUGUUA | GGGCACAUAACAU |
| | 1_N001 | UAGUUAUGAACAGAGGAGACAUAA | AACGGUUAUGUG |
| | | CAUGAACAAGUCACAUAACCGCAA | ACGGAUAGUACUA |
| | | ACUAAACCUGGCGGCAGCGCAA | AUUGAAA (SEQ |
| | | (SEQ ID NO: 15) | ID NO: 115) |
| 16 | STUR_NN_gen | GGGCGAAGAUGAUACAAUUUGUUA | GGGCACCUCACCU |
| | 1_N009 | UAGUUAUGAACAGAGGAGACAUAA | ACGUGUGAGGUG |
| | | CAUGAACAAGCCACCUCACACUCA | GCAUUGUAUCAUC |
| | | ACAUAACCUGGCGGCAGCGCAA | UUCGUAU (SEQ |
| | | (SEQ ID NO: 16) | ID NO: 116) |
| 17 | STUR_WW_ge | GGGAUACUUUCAAACUUAUUGUUA | GGGACACUAAUAC |
| | n1_N016 | UAGUUAUGAACAGAGGAGACAUAA | UACGAUUAGUGU |
| | | CAUGAACAAUCACACUAAUCGACU | GAUAAGUUUGAA |
| | | GAAUAACCUGGCGGCAGCGCAA | AGUAUAAG (SEQ |
| | | (SEQ ID NO: 17) | ID NO: 117) |
| 18 | STUR_WW_ge | GGGUACCUUCAAAUUCCAUUGUUA | GGGACCUCACUUA |
| | n1_N020 | UAGUUAUGAACAGAGGAGACAUAA | CAUUAGUGAGGU |
| | | CAUGAACAAUCACCUCACUAACAU | GAUGGAAUUUGA |
| | | UAUCAACCUGGCGGCAGCGCAA | AGGUAAUG (SEQ |
| | | (SEQ ID NO: 18) | ID NO: 118) |
| 19 | STUR_NN_gen | GGGAUCAAUCAAUUCUACUUGUUA | GGGACAUAAACAU |
| | 1_N011 | UAGUUAUGAACAGAGGAGACAUAA | AGAGGUUUAUGU |
| | | CAUGAACAAUCACAUAAACCUAAG | GAGUAGAAUUGA |
| | | AACUAACCUGGCGGCAGCGCAA | UUGAUAAG (SEQ |
| | | (SEQ ID NO: 19) | ID NO: 119) |
| 20 | STUR_WW_gen | GGGAUUCACUUACAAGAUUUGUUA | GGGACAUAGACAA |
| | 1_N018 | UAGUUAUGAACAGAGGAGACAUAA | UUUGGUCUAUGU |
| | | CAUGAACAAUCACAUAGACCAUAG | GAAUCUUGUAAG |
| | | AACGAACCUGGCGGCAGCGCAA | UGAAUAUG (SEQ |
| | | (SEQ ID NO: 20) | ID NO: 120) |
| 21 | STUR_WW_gen | GGGAUGUGAUUACUAGAUUUGUUA | GGGC GAACAAGAA |
| | 1_N012 | UAGUUAUGAACAGAGGAGACAUAA | CAUGCUUGUUCGG |
| | | CAUGAACAAUCCGAACAAGCAAUA | AAUCUAGUAAUCA |
| | | CAAGAACCUGGCGGCAGCGCAA | CAUCUA (SEQ |
| | | (SEQ ID NO: 21) | ID NO: 121) |
| 22 | STUR_WW_gen | GGGUCCAUAUACUUCAAUUUGUUA | GGGAACCUAAUGU |
| | 1_N009 | UAGUUAUGAACAGAGGAGACAUAA | ACGAAUUAGGUU |
| | | CAUGAACAAUCAACCUAAUUCUGA | GAAUUGAAGUAU |
| | | GAUAAACCUGGCGGCAGCGCAA | AUGGAAUC (SEQ |
| | | (SEQ ID NO: 22) | ID NO: 122) |
| 23 | STUR_WW_gen | GGGCAAUUCCUAAUCCUAUUGUUA | GGGCAAAUAGAU |
| | 1_N023 | UAGUUAUGAACAGAGGAGACAUAA | AGAGCUCUAUUUG |
| | | CAUGAACAAUCCAAAUAGAGCAAA | GAUAGGAUUAGG |
| | | GCAGAACCUGGCGGCAGCGCAA | AAUUGAAA (SEQ |
| | | (SEQ ID NO: 23) | ID NO: 123) |
| 24 | STUR_NN_gen | GGGACAAAUCAGAUAAACUUGUUA | GGGCAAUAGAUCA |
| | 1_N008 | UAGUUAUGAACAGAGGAGACAUAA | AACUAUCUAUUGG |
| | | CAUGAACAAGCCAAUAGAUAGAAC | CGUUUAUCUGAUU |
| | | ACGAAACCUGGCGGCAGCGCAA | UGUAUG (SEQ |
| | | (SEQ ID NO: 24) | ID NO: 124) |
| 25 | STUR_WW_gen | GGGAAUCCAAGUUAUCAUUUGUUA | GGGCACAUAAACU |
| | 1_N011 | UAGUUAUGAACAGAGGAGACAUAA | AAUCUUUAUGUG |
| | | CAUGAACAAUACACAUAAAGACCU | UAAUGAUAACUU |
| | | CAUAAACCUGGCGGCAGCGCAA | GGAUUGAA (SEQ |
| | | (SEQ ID NO: 25) | ID NO: 125) |
| 26 | STUR_WW_gen | GGGAUGAUCGAAACGAAUUUGUUA | GGGCAAGAAUAAC |
| | 1_N014 | UAGUUAUGAACAGAGGAGACAUAA | AUUCUAUUCUUGU |
| | | CAUGAACAAUACAAGAAUAGACAA | AAUUCGUUUCGAU |
| | | UAGAAACCUGGCGGCAGCGCAA | CAUAUG (SEQ |
| | | (SEQ ID NO: 26) | ID NO: 126) |
| 27 | STUR_WW_gen | GGGAAUGAAGAUAGUGAUUUGUUA | GGGCGAAUAAGA |
| | 1_N003 | UAGUUAUGAACAGAGGAGACAUAA | AUGCGCUUAUUCG |
| | | CAUGAACAAUCCGAAUAAGCGAAC | GAAUCACUAUCUU |
| | | UAAGAACCUGGCGGCAGCGCAA | CAUUAAC (SEQ |
| | | (SEQ ID NO: 27) | ID NO: 127) |
| 28 | STUR_WW_gen | GGGAUCUCACCGUGUCUAUUGUUA | GGGAACAUACAAA |
| | 1_N017 | UAGUUAUGAACAGAGGAGACAUAA | UUGAUGUAUGUU |
| | | CAUGAACAAUCAACAUACAUCAUA | GAUAGACACGGUG |
| | | CAAGAACCUGGCGGCAGCGCAA | AGAUAGA (SEQ |
| | | (SEQ ID NO: 28) | ID NO: 128) |
| 29 | STUR_WW_gen | GGGACUCCAAUUGACGAUUUGUUA | GGGCAUAUAGAA |
| | 1_N005 | UAGUUAUGAACAGAGGAGACAUAA | UAGAGUCUAUAU |
| | | CAUGAACAAUACAUAUAGACUAAA | GUAAUCGUCAAUU |
| | | CAUAAACCUGGCGGCAGCGCAA | GGAGUAGA (SEQ |
| | | (SEQ ID NO: 29) | ID NO: 129) |
| 30 | STUR_NN_gen | GGGUAAUGAAGUGAAUACUUGUUA | GGGCAAAUGAUCA |
| | 1_N004 | UAGUUAUGAACAGAGGAGACAUAA | UCGUAUCAUUUGG |
| | | CAUGAACAAGCCAAAUGAUACUAA | CGUAUUCACUUCA |
| | | ACCGAACCUGGCGGCAGCGCAA | UUAACA (SEQ |
| | | (SEQ ID NO: 30) | ID NO: 130) |
| 31 | STUR_NN_gen | GGGUCCCAUUAUCUUACCUUGUUA | GGGCUACACUAAA |
| | 1_N016 | UAGUUAUGAACAGAGGAGACAUAA | CAGGUAGUGUAG |
| | | CAUGAACAAGCCUACACUACCAUU | GCGGUAAGAUAA |
| | | CCCGAACCUGGCGGCAGCGCAA | UGGGAGUA (SEQ |
| | | (SEQ ID NO: 31) | ID NO: 131) |
| 32 | STUR_WW_gen | GGGCGAUAUGAGUAAAGAUUGUUA | GGGCAAUAGAAA |
| | 1_N006 | UAGUUAUGAACAGAGGAGACAUAA | UACCGUUCUAUUG |
| | | CAUGAACAAUGCAAUAGAACGAUA | CAUCUUUACUCAU |
| | | CAAGAACCUGGCGGCAGCGCAA | AUCGAUU (SEQ |
| | | (SEQ ID NO: 32) | ID NO: 132) |
| 33 | STUR_WW_gen | GGGACUACUCCAUUCUAUUUGUUA | GGGCAUACCUCUA |
| | 1_N024 | UAGUUAUGAACAGAGGAGACAUAA | CAAUGAGGUAUG |
| | | CAUGAACAAUCCAUACCUCAUACA | GAAUAGAAUGGA |
| | | ACGAAACCUGGCGGCAGCGCAA | GUAGUAAG (SEQ |
| | | (SEQ ID NO: 33) | ID NO: 133) |
| 34 | STUR_WW_gen | GGGCGACUUAAACUACUAUUGUUA | GGGACUCCACGAA |
| | 1_N021 | UAGUUAUGAACAGAGGAGACAUAA | UAUCCGUGGAGUG |
| | | CAUGAACAAUCACUCCACGGAAAC | AUAGUAGUUUAA |
| | | UACUAACCUGGCGGCAGCGCAA | GUCGUAG (SEQ |
| | | (SEQ ID NO: 34) | ID NO: 134) |
| 35 | STUR_WW_gen | GGGAUGUGAACUUAAAGAUUGUUA | GGGCGAAAUACAA |
| | 1_N013 | UAGUUAUGAACAGAGGAGACAUAA | UACCGUAUUUCGC |
| | | CAUGAACAAUGCGAAAUACGGAAA | AUCUUUAAGUUCA |
| | | UAGAAACCUGGCGGCAGCGCAA | CAUCGA (SEQ ID |
| | | (SEQ ID NO: 35) | NO: 135) |
| 36 | STUR_NN_gen | GGGCAAUCCUAUUCAUCAUUGUUA | GGGCUCAACAUAA |
| | 1_N018 | UAGUUAUGAACAGAGGAGACAUAA | CAUGAUGUUGAG |
| | | CAUGAACAAUUCUCAACAUCAAUC | AAUGAUGAAUAG |
| | | ACUCAACCUGGCGGCAGCGCAA | GAUUGAGA (SEQ |
| | | (SEQ ID NO: 36) | ID NO: 136) |
| 37 | STUR_WW_gen | GGGUAAGAAUUAGAAUCAUUGUUA | GGGAGCGAACAAU |
| | 1_N001 | UAGUUAUGAACAGAGGAGACAUAA | AACGUGUUCGCUG |
| | | CAUGAACAAUCAGCGAACACGAAA | AUGAUUCUAAUUC |
| | | CAUAAACCUGGCGGCAGCGCAA | UUAACA (SEQ |
| | | (SEQ ID NO: 37) | ID NO: 137) |
| 38 | STUR_NN_gen | GGGUCAAUCCGAUAAUCUUUGUUA | GGGACUCCAAGAU |
| | 1_N022 | UAGUUAUGAACAGAGGAGACAUAA | AAGCCUUGGAGUA |
| | | CAUGAACAAUUACUCCAAGGCAAU | AAGAUUAUCGGA |
| | | GAUAAACCUGGCGGCAGCGCAA | UUGAAUG (SEQ |
| | | (SEQ ID NO: 38) | ID NO: 138) |
| 39 | STUR_WW_gen | GGGAAUGAAUGAUGAGAUUUGUUA | GGGAGCGAACGCG |
| | 1_N007 | UAGUUAUGAACAGAGGAGACAUAA | AACUCGUUCGCUC |
| | | CAUGAACAAUGAGCGAACGAGCAA | AAUCUCAUCAUUC |
| | | UGGAAACCUGGCGGCAGCGCAA | AUUAAG (SEQ |
| | | (SEQ ID NO: 39) | ID NO: 139) |
| 40 | STUR_NN_gen | GGGAUUUAGAAGUAAGUAUUGUUA | GGGCGAAGAUAG |
| | 1_N006 | UAGUUAUGAACAGAGGAGACAUAA | AUACAUAUCUUCG |
| | | CAUGAACAAUUCGAAGAUAUGGGA | AAUACUUACUUCU |
| | | CGAUAACCUGGCGGCAGCGCAA | AAAUCUA (SEQ |
| | | (SEQ ID NO: 40) | ID NO: 140) |
| 41 | STUR_WW_gen | GGGAUUUACUUAGAUACUUUGUUA | GGGACAUAUACUU |
| | 1_N004 | UAGUUAUGAACAGAGGAGACAUAA | UCAUGUAUAUGU |
| | | CAUGAACAAUUACAUAUACAUACG | AAAGUAUCUAAG |
| | | AACGAACCUGGCGGCAGCGCAA | UAAAUGAA (SEQ |
| | | (SEQ ID NO: 41) | ID NO: 141) |
| 42 | STUR_WW_gen | GGGCGAAUUGAAAUGAAAUUGUUA | GGGACGGAAUAAC |
| | 1_N015 | UAGUUAUGAACAGAGGAGACAUAA | ACUCUAUUCCGUC |
| | | CAUGAACAAUGACGGAAUAGAAAC | AUUUCAUUUCAAU |
| | | UAAGAACCUGGCGGCAGCGCAA | UCGUAA (SEQ |
| | | (SEQ ID NO: 42) | ID NO: 142) |
| 43 | STUR_WW_gen | GGGCAUUUACCCUACUAUUUGUUA | GGGCACUCCAAUA |
| | 1_N019 | UAGUUAUGAACAGAGGAGACAUAA | GGAUUUGGAGUG |
| | | CAUGAACAAUACACUCCAAAUCAA | UAAUAGUAGGGU |
| | | AGAUAACCUGGCGGCAGCGCAA | AAAUGAGA (SEQ |
| | | (SEQ ID NO: 43) | IDN0 43) |
| 44 | STUR_WW_gen | GGGUAUUACUUACCGAUAUUGUUA | GGGACUCCAACUA |
| | 1_N002 | UAGUUAUGAACAGAGGAGACAUAA | AACCGUUGGAGUG |
| | | CAUGAACAAUCACUCCAACGGAAC | AUAUCGGUAAGU |
| | | AAUCAACCUGGCGGCAGCGCAA | AAUAGAA (SEQ |
| | | (SEQ ID NO: 44) | ID NO: 144) |
| 45 | STUR_WW_gen | GGGCUCAUACUUCACUAUUUGUUA | GGGCACCUAACUA |
| | 1_N022 | UAGUUAUGAACAGAGGAGACAUAA | ACAUGUUAGGUG |
| | | CAUGAACAAUACACCUAACAUAAU | UAAUAGUGAAGU |
| | | CACUAACCUGGCGGCAGCGCAA | AUGAGAUG (SEQ |
| | | (SEQ ID NO: 45) | ID NO: 145) |
| 46 | STUR_NN_gen | GGGAUCUCCAUCUAUCCAUUGUUA | GGGCAUUAAGUU |
| | 1_N005 | UAGUUAUGAACAGAGGAGACAUAA | AAGUUACUUAAU |
| | | CAUGAACAAUCCAUUAAGUAACAA | GGAUGGAUAGAU |
| | | GCAGAACCUGGCGGCAGCGCAA | GGAGAUGAU |
| | | (SEQ ID NO: 46) | (SEQ ID |
| | | | NO: 146) |
| 47 | STUR_NN_gen | GGGUAGAUUAAGAGUGAUUUGUUA | GGGACCUAAACUA |
| | 1_N014 | UAGUUAUGAACAGAGGAGACAUAA | ACGCGUUUAGGUC |
| | | CAUGAACAAUGACCUAAACGCACG | AAUCACUCUUAAU |
| | | AACUAACCUGGCGGCAGCGCAA | CUACUA (SEQ |
| | | (SEQ ID NO: 47) | ID NO: 147) |
| 48 | STUR_NN_gen | GGGCAAUCUCCAAUACGUUUGUUA | GGGACUUAAACGA |
| | 1_N017 | UAGUUAUGAACAGAGGAGACAUAA | AACGGUUUAAGU |
| | | CAUGAACAAGCACUUAAACCGAAU | GCACGUAUUGGAG |
| | | AACUAACCUGGCGGCAGCGCAA | AUUGGGA (SEQ |
| | | (SEQ ID NO: 48) | ID NO: 148) |

TABLE 2
1-Input (“1-bit”) NOR Devices

| Device | | | Trigger RNA |
|---|---|---|---|
| Number | Device Name | 5′-3′ Switch RNA Sequence | Sequence |
| 1 | STUR_NOR_ | GGGAUCAAAGUAUACAAGGGUAGGGUAGGGUUA | GGGCUGCAGGUG |
| | Gen3_Hpin5 | UCGCGACACUAAUAGUGUAGUGUCGGAUAAGUG | ACAGAUAUCACC |
| | | AAUAGUAGUCGAUAACCCUACCCUACCGCCUCA | UGCAGCGAUAAC |
| | | AUUCGAGGCAAGACUACUAUUCACUUAUUGUUA | CCUACCCUACCCU |
| | | UAGUUAUGAACAGAGGAGACAUAACAUGAACAA | UGUAUACUUUGA |
| | | UCCGACACUACAAUUUCGAAACCUGGCGGCAGC | U |
| | | GCAAA (SEQ ID NO: 49) | (SEQ ID NO: 149) |
| 2 | STUR_NOR_ | GGGUCUUAUGUGUAUGUUUCUGUCUUGUCUUUA | GGGACAAGGACU |
| | Gen2_Hpin1 | UCUCGACACUAAUAGUGUAGUGUCGGAUAAGUG | CUGAAGUGAGUC |
| | | AAUAGUAGUAGAUAAAGACAAGACAGAGCCUCA | CUUGUAGAUAAA |
| | | AUUCGAGGCAAGACUACUAUUCACUUAUUGUUA | GACAAGACAGAA |
| | | UAGUUAUGAACAGAGGAGACAUAACAUGAACAA | ACAUACACAUAA |
| | | UCCGACACUACAAUUUCGAAACCUGGCGGCAGC | GA |
| | | GCAAA (SEQ ID NO: 50) | (SEQ ID NO: 150) |
| 3 | STUR_NOR_ | GGGAGUAAAGUAUAGAACGCUUGAUGUUGAUGU | GGGUGACAUAUC |
| | Gen3_Hpin7_ | UAGCGACACUAAUAGUGUAGUGUCGGAUAAGUG | CAAGAGCGGAUA |
| | mut | AAUAUAGUCUAACAUCAACAUCAAGCGCCUCAA | UGUCACUAACAU |
| | | UUCGAGGCAAGACUACUAUUCACUUAUUGUUAU | CAACAUCAAGCG |
| | | AGUUAUGAACAGAGGAGACAUAACAUGAACAAU | UUCUAUACUUUA |
| | | CCGACACUACAAUUUCGAAACCUGGCGGCAGCG | CU |
| | | CAAA (SEQ ID N0: 51) | (SEQ ID NO: 151) |
| 4 | STUR_NOR_ | GGGUCGUAUAAGUUUAAGUCUGUUUCUGUCUGU | GGGACCUCAAUU |
| | Gen2_Hpin9_ | UCUCGACACUAAUAGUGUAGUGUCGGAUAAGUG | CAUCUGGGAAUU |
| | mutRepstem | AAUAGUAGUAGAACAGACAGAAACAGAGCCUCA | GAGGUAGAACAG |
| | | AUUCGAGGCAAGACUACUAUUCACUUAUUGUUA | ACAGAAACAGAC |
| | | AGUUAUGAACAGAGGAGACAUAACAUGAACAAU | UUAAACUUAUAC |
| | | CCGACACUACAAUUUCGAAACCUGGCGGCAGCG | GA |
| | | ACAA (SEQ ID NO: 52) | (SEQ ID NO: 152) |
| 5 | STUR_NOR_ | GGGACUAAGACUAAAUGAUGUAUAGAUUGAAGA | GGGACGUGCGCA |
| | Gen3_Hpin11 | UUGCGACACUAAUAGUGUAGUGUCGGAUAAGUG | GCUCCUACUGCGC |
| | | AAUAGUAGUCAAUCUUCAAUCUAUACAGCCUCA | ACGUCAAUCUUC |
| | | AUUCGAGGCAAGACUACUAUUCACUUAUUGUUA | AAUCUAUACAUC |
| | | UAGUUAUGAACAGAGGAGACAUAACAUGAACAA | AUUUAGUCUUAG |
| | | UCCGACACUACAAUUUCGAAACCUGGCGGCAGC | U |
| | | GCAAA (SEQ ID NO: 53) | (SEQ ID NO: 153) |
| 6 | STUR_NOR_ | GGGUUCUUAGUUUAUGUAGGUCUGUUAUCUUGU | GGGUGACCUGAA |
| | Gen2_Hpin13 | UCUCGACACUAAUAGUGUAGUGUCGGAUAAGUG | CCGAACUGUUCA |
| | | AAUAGUAGUAGAACAAGAUAACAGACCGCCUCA | GGUCAAGAACAA |
| | | AUUCGAGGCAAGACUACUAUUCACUUAUUGUUA | GAUAACAGACCU |
| | | UAGUUAUGAACAGAGGAGACAUAACAUGAACAA | ACAUAAACUAAG |
| | | UCCGACACUACAAUUUCGAAACCUGGCGGCAGC | AA |
| | | GCAAA (SEQ ID NO: 54) | (SEQ ID NO: 154) |
| 7 | STUR_NOR_ | GGGACGACUUAAGAUUACGGUAUGAGUUUAGAU | GGGAGUGCCACA |
| | Gen3_Hpin6_ | UUGCGACACUAAUAGUGUAGUGUCGGAUAAGUA | GGCUCGACUGUG |
| | mut | AUAGUAGUCAAAUCUAAACUCAUACCGCCUCAA | GCACUCAAAUCU |
| | | UUCGAGGCAAGACUACUAUUCACUUAUUGUUAU | AAACUCAUACCG |
| | | AGUUAUGAACAGAGGAGACAUAACAUGAACAAU | UAAUCUUAAGUC |
| | | CCGACACUACAAUUUCGAAACCUGGCGGCAGCGC | GU |
| | | AAA (SEQ ID NO: 55) | (SEQ ID NO: 155) |
| 8 | STUR_NOR_ | GGGUUAGGUAUGUGUAUUGAUUGAUUUGAUGCU | GGGACCCUACUCC |
| | Gen2_Hpin12 | UGUCGACACUAAUAGUGUAGUGUCGGAUAAGUG | AGCACAGGAGU A |
| | | AAUAGUAGUACAAGCAUCAAAUCAAUCGCCUCA | GGGUACAAGCAU |
| | | AUUCGAGGCAAGACUACUAUUCACUUAUUGUUA | CAAAUCAAUCAA |
| | | UAGUUAUGAACAGAGGAGACAUAACAUGAACAA | UACACAUACCUA |
| | | UCCGACACUACAAUUUCGAAACCUGGCGGCAGC | A |
| | | GCAAA (SEQ ID NO: 56) | (SEQ ID NO: 156) |
| 9 | STUR_NOR_ | GGGAGUAAGGUAAGGUGAGUGCGUUGUCUUUAG | GGGUCACUACUG |
| | Gen2_Hpin16 | UCUCGACACUAAUAGUGUAGUGUCGGAUAAGUG | GGAUAUACCAGU |
| | | AAUAGUAGUAGACUAAAGACAACGCACGCCUCA | AGUGAAGACUAA |
| | | AUUCGAGGCAAGACUACUAUUCACUUAUUGUUA | AGACAACGCACUC |
| | | UAGUUAUGAACAGAGGAGACAUAACAUGAACAA | ACCUUACCUUACU |
| | | UCCGACACUACAAUUUCGAAACCUGGCGGCAGC | (SEQ ID NO: 157) |
| | | GCAAA (SEQ ID NO: 57) | |
| 10 | STUR_NOR | GGGUCUGUUUAUGUGUAUGUGUCUGUCUUGUCU | GGGACGUUAGUC |
| | Gen2_Hpin3 | GCUCGACACUAAUAGUGUAGUGUCGGAUAAGUG | GAUGUCGCGACU |
| | | AAUAGUAGUAGCAGACAAGACAGACACGCCUCA | AACGUAGCAGAC |
| | | AUUCGAGGCAAGACUACUAUUCACUUAUUGUUA | AAGACAGACACA |
| | | UAGUUAUGAACAGAGGAGACAUAACAUGAACAA | UACACAUAAACA |
| | | UCCGACACUACAAUUUCGAAACCUGGCGGCAGC | GA |
| | | GCAAA (SEQ ID NO: 58) | (SEQ ID NO: 158) |
| 11 | STUR_NOR_ | GGGAGCUUAAUGAUAGAAUGCGUUGAUGUUAGU | GGGUCGGUGCGU |
| | GGen3_Hpin3_ | AAGCGACACUAAUAGUGUAGUGUCGGAUAAGUG | UAACGGCAACGC |
| | mut | AAUAGUAGUCUUACUAACAUCAACGCAGCCUCA | ACCGACUUACUA |
| | | AUUCGAGGCAAGACUACUAUUCACUUAUUGUUA | ACAUCAACGCAU |
| | | UAGUUAUGAACAGAGGAGACAUAACAUGAACAA | UCUAUCAUUAAG |
| | | UCCGACACUACAAUUUCGAAACCUGGCGGCAGC | CU |
| | | GCAAA (SEQ ID NO: 59) | (SEQ ID NO: 159) |
| 12 | STUR_NOR_ | GGGAGUAAAGUAUAGAACGCUUGAUGUUGAUGU | GGGUGACAUAUC |
| | Gen3_Hpin7 | UAGCGACACUAAUAGUGUAGUGUCGGAUAAGUG | CAAGAGCGGAUA |
| | | AAUAGUAGUCUAACAUCAACAUCAAGCGCCUCA | UGUCACUAACAU |
| | | AUUCGAGGCAAGACUACUAUUCACUUAUUGUUA | CAACAUCAAGCG |
| | | UAGUUAUGAACAGAGGAGACAUAACAUGAACAA | UUCUAUACUUUA |
| | | UCCGACACUACAAUUUCGAAACCUGGCGGCAGC | CU |
| | | GCAAA (SEQ ID NO: 60) | (SEQ ID NO: 160) |
| 13 | STUR_NOR_ | GGGAGUUGUAUGUGUAUGGUCUGUCUUGUUCUA | GGGACGCGCUAG |
| | Gen2_Hpin2 | UCUCGACACUAAUAGUGUAGUGUCGGAUAAGUG | CGCAUCGGCUAGC |
| | | AAUAGUAGUAGAUAGAACAAGACAGACGCCUCA | GCGUAGAUAGAA |
| | | AUUCGAGGCAAGACUACUAUUCACUUAUUGUUA | CAAGACAGACCA |
| | | UAGUUAUGAACAGAGGAGACAUAACAUGAACAA | UACACAUACAAC |
| | | UCCGACACUACAAUUUCGAAACCUGGCGGCAGC | U |
| | | GCAAA (SEQ ID NO: 61) | (SEQ ID NO: 161) |
| 14 | STUR_NOR_ | GGGAUGAUAAAGAUUGAUGCUGUUAUGUUGAUU | GGGAGCCAUGAU |
| | Gen3_Hpin4_ | UAGCGACACUAAUAGUGUAGUGUCGGAUAAGGA | CGCCCAGGAUCAU |
| | mut | AUAGUAGUCUAAAUCAACAUAACAGCGCCUCAA | GGCUCUAAAUCA |
| | | UUCGAGGCAAGACUACUAUUCACUUAUUGUUAU | ACAUAACAGCAU |
| | | AGUUAUGAACAGAGGAGACAUAACAUGAACAAU | CAAUCUUUAUCA |
| | | CCGACACUACAAUUUCGAAACCUGGCGGCAGCGC | U |
| | | AAA (SEQ ID NO: 62) | (SEQ ID NO: 162) |
| 15 | STUR_NOR_ | GGGAUGAUAAAGAUUGAUGCUGUUAUGUUGAUU | GGGAGCCAU GAU |
| | Gen3_Hpin4_ | UAGCGACACUAAUAGUGUAGUGUCGGAUAAUGA | CGCCCAGGAUCAU |
| | mut | AUAGUAGUCUAAAUCAACAUAACAGCGCCUCAA | GGCUCUAAAUCA |
| | | UUCGAGGCAAGACUACUAUUCACUUAUUGUUAU | ACAUAACAGCAU |
| | | AGUUAUGAACAGAGGAGACAUAACAUGAACAAU | CAAUCUUUAUCA |
| | | CCGACACUACAAUUUCGAAACCUGGCGGCAGCG | U |
| | | CAAA (SEQ ID NO: 63) | (SEQ ID NO: 163) |
| 16 | STUR_NOR_ | GGGAGUAAAGUAAAGGUGUCGUUGUAUGUUCGU | GGGACACCGGAG |
| | Gen2_Hpin6 | UCUCGACACUAAUAGUGUAGUGUCGGAUAAGUG | GCGUGCACCUCCG |
| | | AAUAGUAGUAGAACGAACAUACAACGAGCCUCA | GUGUAGAACGAA |
| | | AUUCGAGGCAAGACUACUAUUCACUUAUUGUUA | CAUACAACGACAC |
| | | UAGUUAUGAACAGAGGAGACAUAACAUGAACAA | CUUUACUUUACU |
| | | UCCGACACUACAAUUUCGAAACCUGGCGGCAGC | (SEQ ID NO: 164) |
| | | GCAAA (SEQ ID NO: 64) | |
| 17 | STUR_NOR | GGGUCUAGUGUCGUCGUUGUCUUGUAUGGUUCU | GGGACGAUAUAC |
| | Gen2_Hpin15 | UCUCGACACUAAUAGUGUAGUGUCGGAUAAGUG | GGGUCUACGUAU |
| | | AAUAGUAGUACAAGAACCAUACAAGACGCCUCA | AUCGUACAAGAA |
| | | AUUCGAGGCAAGACUACUAUUCACUUAUUGUUA | CCAUACAAGACA |
| | | UAGUUAUGAACAGAGGAGACAUAACAUGAACAA | ACGACGACACUA |
| | | UCCGACACUACAAUUUCGAAACCUGGCGGCAGC | GA |
| | | GCAAA (SEQ ID NO: 65) | (SEQ ID NO: 165) |
| 18 | STUR NOR | GGGUUAGUAUUGUAGUUGUCUGUAUUUGUCUCU | GGGCAGGGUACU |
| | Gen2_Hpin18 | GCUCGACACUAAUAGUGUAGUGUCGGAUAAGUG | UGUACUGAAGUA |
| | | AAUAGUAGUAGCAGAGACAAAUACAGAGCCUCA | CCCUGAGCAGAG |
| | | AUUCGAGGCAAGACUACUAUUCACUUAUUGUUA | ACAAAUACAGAC |
| | | UAGUUAUGAACAGAGGAGACAUAACAUGAACAA | AACUACAAUACU |
| | | UCCGACACUACAAUUUCGAAACCUGGCGGCAGC | AA |
| | | GCAAA (SEQ ID NO: 66) | (SEQ ID NO: 166) |
| 19 | STUR_NOR_ | GGGAUCAAAGUAUACAAGGGUAGGGUAGGGUUA | GGGCUGCAGGUG |
| | Gen3_Hpin5_ | UCGCGACACUAAUAGUGUAGUGUCGGAUAAGUG | ACAGAUAUCACC |
| | rnutA | AUAGUAGUCGAUAACCCUACCCUACCGCCUCAA | UGCAGCGAUAAC |
| | | UUCGAGGCAAGACUACUAUUCACUUAUUGUUAU | CCUACCCUACCCU |
| | | AGUUAUGAACAGAGGAGACAUAACAUGAACAAU | UGUAUACUUUGA |
| | | CCGACACUACAAUUUCGAAACCUGGCGGCAGCG | U |
| | | CAAA (SEQ ID NO: 67) | (SEQ ID NO: 167) |
| 20 | STUR_NOR_ | GGGACUGGAUACUUAUACGGUUCUGUAGGUUCU | GGGCCAUACCUG |
| | Gen3_ | UAGCGACACUAAUAGUGUAGUGUCGGAUAAGUU | GCAACUACCAGG |
| | Hpin10_ | AAUAGUAGUCUAAGAACCUACAGAACCGCCUCA | UAUGGCUAAGAA |
| | mut | AUUCGAGGCAAGACUACUAUUCACUUAUUGUUA | CCUACAGAACCGU |
| | | UAGUUAUGAACAGAGGAGACAUAACAUGAACAA | AUAAGUAUCCAG |
| | | UCCGACACUACAAUUUCGAAACCUGGCGGCAGC | U |
| | | GCAAA (SEQ ID NO: 68) | (SEQ ID NO: 168) |
| 21 | STUR_NOR_ | GGGUUAGUGUAUGGUAUGUCGUUCUUGUUCUUA | GGGAGCCGUGCA |
| | Gen2_Hpin4 | UCUCGACACUAAUAGUGUAGUGUCGGAUAAGUG | GCGUGCACUGCAC |
| | | AAUAGUAGUAGAUAAGAACAAGAACGAGCCUCA | GGCUAGAUAAGA |
| | | AUUCGAGGCAAGACUACUAUUCACUUAUUGUUA | ACAAGAACGACA |
| | | UAGUUAUGAACAGAGGAGACAUAACAUGAACAA | UACCAUACACUA |
| | | UCCGACACUACAAUUUCGAAACCUGGCGGCAGC | A |
| | | GCAAA (SEQ ID NO: 69) | (SEQ ID NO: 169) |
| 22 | STUR_NOR_ | GGGACGUACUAAUUGAUUUCGUCUUGUCUUGUA | GGGUUCCCGUCAC |
| | Gen3_Hpin8 | UUGCGACACUAAUAGUGUAGUGUCGGAUAAGUG | UGGUACGUGACG |
| | | AAUAGUAGUCAAUACAAGACAAGACGAGCCUCA | GGAACAAUACAA |
| | | AUUCGAGGCAAGACUACUAUUCACUUAUUGUUA | GACAAGACGAAA |
| | | UAGUUAUGAACAGAGGAGACAUAACAUGAACAA | UCAAUUAGUACG |
| | | UCCGACACUACAAUUUCGAAACCUGGCGGCAGC | U |
| | | GCAAA (SEQ ID NO: 70) | (SEQ ID NO: 170) |
| 23 | STUR_NOR_ | GGGAGUGUAUUGUAUGUGUCUUGUUCUUGUUCG | GGGACCUGCUCAC |
| | Gen2_Hpin5 | UCUCGACACUAAUAGUGUAGUGUCGGAUAAGUG | CUUCAUGUGAGC |
| | | AAUAGUAGUAGACGAACAAGAACAAGAGCCUCA | AGGUAGACGAAC |
| | | AUUCGAGGCAAGACUACUAUUCACUUAUUGUUA | AAGAACAAGACA |
| | | UAGUUAUGAACAGAGGAGACAUAACAUGAACAA | CAUACAAUACAC |
| | | UCCGACACUACAAUUUCGAAACCUGGCGGCAGC | U |
| | | GCAAA (SEQ ID NO: 71) | (SEQ ID NO: 171) |
| 24 | STUR_NOR | GGGUCCUGUGUCUUUAUGUCUUGUUCUGUUUAU | GGGACCGUAACG |
| | Gen2_Hpin14 | CUUCGACACUAAUAGUGUAGUGUCGGAUAAGUG | AAAUCCAUCGUU |
| | | AAUAGUAGUAAGAUAAACAGAACAAGAGCCUCA | ACGGUAAGAUAA |
| | | AUUCGAGGCAAGACUACUAUUCACUUAUUGUUA | ACAGAACAAGAC |
| | | UAGUUAUGAACAGAGGAGACAUAACAUGAACAA | AUAAAGACACAG |
| | | UCCGACACUACAAUUUCGAAACCUGGCGGCAGC | GA |
| | | GCAAA (SEQ ID NO: 72) | (SEQ ID NO: 172) |
| 25 | STUR_NOR_ | GGGUCUGUGUAUUUCUGUUCUUAUGUCUUGUCU | GGGACAUCAGAG |
| | Gen2_Hpin17 | UAUCGACACUAAUAGUGUAGUGUCGGAUAAGUG | ACUAGUAUCUCU |
| | mut | AAAGUAGUAUAAGACAAGACAUAAGAGCCUCAA | GAUGUAUAAGAC |
| | | UUCGAGGCAAGACUACUAUUCACUUAUUGUUAU | AAGACAUAAGAA |
| | | AGUUAUGAACAGAGGAGACAUAACAUGAACAAU | CAGAAAUACACA |
| | | CCGACACUACAAUUUCGAAACCUGGCGGCAGCG | GA |
| | | CAAA (SEQ ID NO: 73) | (SEQ ID NO: 173) |
| 26 | STUR_NOR_ | GGGAUGUAUGUGUAAUGGUCUGUUUAUGUAUGU | GGGAGUCACGCG |
| | Gen2_Hpin10 | UCUCGACACUAAUAGUGUAGUGUCGGAUAAGUG | CUACGUUGCGCG |
| | | AAUAGUAGUAGAACAUACAUAAACAGAGCCUCA | UGACUAGAACAU |
| | | AUUCGAGGCAAGACUACUAUUCACUUAUUGUUA | ACAU AAACAGAC |
| | | UAGUUAUGAACAGAGGAGACAUAACAUGAACAA | CAUUACACAUAC |
| | | UCCGACACUACAAUUUCGAAACCUGGCGGCAGC | AU |
| | | GCAAA (SEQ ID NO: 74) | (SEQ ID NO: 174) |
| 27 | STUR_NOR_ | GGGAAUCUUAAUCGUAUAGGUCUUUGUCUUGUU | GGGAGUCCUUGA |
| | Gen3_Hpin2 | CUGCGACACUAAUAGUGUAGUGUCGGAUAAGUG | GGUGCGACUCAA |
| | | AAUAGUAGUCAGAACAAGACAAAGACCGCCUCA | GGACUCAGAACA |
| | | AUUCGAGGCAAGACUACUAUUCACUUAUUGUUA | AGACAAAGACCU |
| | | UAGUUAUGAACAGAGGAGACAUAACAUGAACAA | AUACGAUUAAGA |
| | | UCCGACACUACAAUUUCGAAACCUGGCGGCAGC | UU |
| | | GCAAA (SEQ ID NO: 75) | (SEQ ID NO: 175) |
| 28 | STUR_NOR_ | GGGAGUAGUAGUAAAUGUGUCUUGUAUCUUGUU | GGGCCACUUCUG |
| | Gen3_Hpin9 | CUGCGACACUAAUAGUGUAGUGUCGGAUAAGUG | ACU GCGAUCAGA |
| | | AAUAGUAGUCAGAACAAGAUACAAGACGCCUCA | AGUGGCAGAACA |
| | | AUUCGAGGCAAGACUACUAUUCACUUAUUGUUA | AGAUACAAGACA |
| | | UAGUUAUGAACAGAGGAGACAUAACAUGAACAA | CAUUUACUACUA |
| | | UCCGACACUACAAUUUCGAAACCUGGCGGCAGC | CU |
| | | GCAAA (SEQ ID NO: 76) | (SEQ ID NO: 176) |
| 29 | STUR_NOR_ | GGGAGUAAGUUGAAAGUAGGUUAUGUUUAUGUU | GGGACCCUCAUU |
| | Gen2_Hpin11 | GCUCGACACUAAUAGUGUAGUGUCGGAUAAGUG | GCUCUACCAAUG |
| | | AAUAGUAGUAGCAACAUAAACAUAACCGCCUCA | AGGGUAGCAACA |
| | | AUUCGAGGCAAGACUACUAUUCACUUAUUGUUA | UAAACAUAACCU |
| | | UAGUUAUGAACAGAGGAGACAUAACAUGAACAA | ACUUUCAACUUA |
| | | UCCGACACUACAAUUUCGAAACCUGGCGGCAGC | CU |
| | | GCAAA (SEQ ID NO: 77) | (SEQ ID NO: 177) |

TABLE 3
2-Input NOR Devices

| Device Name | 2-input NOR Hairpin BA |
|---|---|
| Switch RNA | GGGUCGUAUAAGUUUAAGUCUGU |
| Sequence | UUCUGUCUGUUCUCGACACUAAU |
| | AGUGUAGUGUCGGAUAAGUGAAU |
| | AGUAGUAGAACAGACAGAAACAG |
| | AGCCUCAAUUCGAGGCAAGUCUU |
| | AUGUGUAUGUUUCUGUCUUGUCU |
| | UUAUCUCGACACUAAUAGUGUAG |
| | UGUCGGAUAAGUGAAUAGUAGUA |
| | GAUAAAGACAAGACAGAGCCUCA |
| | AUUCGAGGCAAGACUACUAUUCA |
| | CUUAUUGUUAUAGUUAUGAACAG |
| | AGGAGACAUAACAUGAACAAUCC |
| | GACACUACAAUUUCGA |
| | (SEQ ID NO: 78) |
| Input A | GGGACCUCAAUUCAUCUGGGAAU |
| (Binds at 5′ | UGAGGUAGAACAGACAGAAACAG |
| site on | ACUUAAACUUAUACGA |
| switch RNA) | (SEQ ID NO: 79) |
| Input B | GGGACAAGGACUCUGAAGUGAGU |
| (Binds at 3′ | CCUUGUAGAUAAAGACAAGACA |
| site on | GAAACAUACACAUAAGA |
| switch RNA) | (SEQ ID NO: 80 |

TABLE 4
2-Input NAND Constructs

| | | Input | |
|---|---|---|---|
| | | RNAs | |
| | | Used | |
| | | (Listed | |
| | | by | |
| | | binding | |
| | | location | |
| | | 5′ to 3′ | |
| Device | Device | on switch | |
| Number | Name | RNA) | Switch RNA Sequence |
| 1 | STUR_NAND_ | F, D | GGGCUCCUAUCACUUUACUU |
| | L11_N008_ | | GUUAUAGUUAUGAACAGAGG |
| | HpFD | | AGACAUAACAUGAACAAGCA |
| | | | CACUAACUACAAAUUCCAAA |
| | | | GCCACAUACUAAUCAGAUCU |
| | | | ACUUGUUAUAGUUAUGAACA |
| | | | GAGGAGACAUAACAUGAACA |
| | | | AGCACCUAACAAGACUAAUC |
| | | | (SEQ ID NO: 81) |
| 2 | STUR_ NAND_ | F, D | GGGCUCCUAUCACUUUACUU |
| | L17_N006_ | | GUUAUAGUUAUGAACAGAGG |
| | HpFD | | AGACAUAACAUGAACAAGCA |
| | | | CACUAACUACAAAUUCACCG |
| | | | CAAAUACAAUAACACUAAUC |
| | | | AGAUCUACUUGUUAUAGUUA |
| | | | UGAACAGAGGAGACAUAACA |
| | | | UGAACAAGCACCUAACAAGA |
| | | | CUAAUC |
| | | | (SEQ ID NO: 82) |
| 3 | STUR_ NAND_ | D, B | GGGACUAAUCAGAUCUACUU |
| | L17_N008_ | | GUUAUAGUUAUGAACAGAGG |
| | HpDB | | AGACAUAACAUGAACAAGCA |
| | | | CCUAACAAGACUAAUCAUAU |
| | | | ACAAUACCUCCAAAUCAAUC |
| | | | AAUUCUACUUGUUAUAGUUA |
| | | | UGAACAGAGGAGACAUAACA |
| | | | UGAACAAUCACAUAAACCUA |
| | | | AGAACU |
| | | | (SEQ ID NO: 83) |
| 4 | STUR_ NAND_ | D, C | GGGACUAAUCAGAUCUACUU |
| | L11_N007_ | | GUUAUAGUUAUGAACAGAGG |
| | HpDC | | AGACAUAACAUGAACAAGCA |
| | | | CCUAACAAGACUAAUCCAAA |
| | | | UCAAAUCACAAUCAAAUACA |
| | | | AAUUGUUAUAGUUAUGAACA |
| | | | GAGGAGACAUAACAUGAACA |
| | | | AUCACAUACAAAGCAAACGA |
| | | | (SEQ ID NO: 84) |
| 5 | STUR_ NAND_ | C, F | GGGACAAUCAAAUACAAAUU |
| | L11_N002_ | | GUUAUAGUUAUGAACAGAGG |
| | HpCF | | AGACAUAACAUGAACAAUCA |
| | | | CAUACAAAGCAAACGAACAA |
| | | | UACCCGACUCCUAUCACUUU |
| | | | ACUUGUUAUAGUUAUGAACA |
| | | | GAGGAGACAUAACAUGAACA |
| | | | AGCACACUAACUACAAAUUC |
| | | | (SEQ ID NO: 85) |
| 6 | STUR_ NAND_ | C, D | GGGACAAUCAAAUACAAAUU |
| | L17_N001_ | | GUUAUAGUUAUGAACAGAGG |
| | HpCD | | AGACAUAACAUGAACAAUCA |
| | | | CAUACAAAGCAAACGAAACA |
| | | | UAAAGCCAACUAAACUAAUC |
| | | | AGAUCUACUUGUUAUAGUUA |
| | | | UGAACAGAGGAGACAUAACA |
| | | | UGAACAAGCACCUAACAAGA |
| | | | CUAAUC |
| | | | (SEQ ID NO: 86) |

TABLE 5
3-Input NAND Constructs

| | | Input | |
|---|---|---|---|
| | | RNAs | |
| | | (Listed | |
| | | by | |
| | | binding | |
| | | location | |
| | | 5' to 3' | |
| | | on | |
| | | switch | Switch RNA |
| Device | Device Name | RNA) | Sequence Used |
| 1 | STUR_NAND_ | C, B, D | GGGACAAUCAAAUACAAAUU |
| | link_17_N0 | | GUUAUAGUUAUGAACAGAGG |
| | 3_003_ | | AGACAUAACAUGAACAAUCA |
| | HpCBD | | CAUACAAAGCAAACGAAAUA |
| | | | CCGCAAAUACCAUAUCAAUC |
| | | | AAUUCUACUUGUUAUAGUUA |
| | | | UGAACAGAGGAGACAUAACA |
| | | | UGAACAAUCACAUAAACCUA |
| | | | AGAACUAAGCCGACAAUACU |
| | | | AACACUAAUCAGAUCUACUU |
| | | | GUUAUAGUUAUGAACAGAGG |
| | | | AGACAUAACAUGAACAAGCA |
| | | | CCUAACAAGACUAAUC |
| | | | (SEQ ID NO: 87) |
| 2 | STUR_NAND_ | B, C, D | GGGAUCAAUCAAUUCUACUU |
| | link_11_N0 | | GUUAUAGUUAUGAACAGAGG |
| | 3_004_ | | AGACAUAACAUGAACAAUCA |
| | HpBCD | | CAUAAACCUAAGAACUCAAU |
| | | | AUCGCAUACAAUCAAAUACA |
| | | | AAUUGUUAUAGUUAUGAACA |
| | | | GAGGAGACAUAACAUGAACA |
| | | | AUCACAUACAAAGCAAACGA |
| | | | CAAAUACAAAUACUAAUCAG |
| | | | AUCUACUUGUUAUAGUUAUG |
| | | | AACAGAGGAGACAUAACAUG |
| | | | AACAAGCACCUAACAAGACU |
| | | | AAUC |
| | | | (SEQ ID NO: 88) |
| 3 | STUR_NAND_ | F, C, D | GGGCUCCUAUCACUUUACUU |
| | link_11_N0 | | GUUAUAGUUAUGAACAGAGG |
| | 3_006_ | | AGACAUAACAUGAACAAGCA |
| | HpFCD | | CACUAACUACAAAUUCGCAA |
| | | | AUACAUCACAAUCAAAUACA |
| | | | AAUUGUUAUAGUUAUGAACA |
| | | | GAGGAGACAUAACAUGAACA |
| | | | AUCACAUACAAAGCAAACGA |
| | | | CAAAUACAAAUACUAAUCAG |
| | | | AUCUACUUGUUAUAGUUAUG |
| | | | AACAGAGGAGACAUAACAUG |
| | | | AACAAGCACCUAACAAGACU |
| | | | AAUC (SEQ ID NO: 89) |
| 4 | STUR_NAND_ | F, D, C | GGGCUCCUAUCACUUUACUU |
| | link_11_N0 | | GUUAUAGUUAUGAACAGAGG |
| | 3_001_ | | AGACAUAACAUGAACAAGCA |
| | HpFDC | | CACUAACUACAAAUUCCAAA |
| | | | GCCACAUACUAAUCAGAUCU |
| | | | ACUUGUUAUAGUUAUGAACA |
| | | | GAGGAGACAUAACAUGAACA |
| | | | AGCACCUAACAAGACUAAUC |
| | | | CAAAUCAAAUCACAAUCAAA |
| | | | UACAAAUUGUUAUAGUUAUG |
| | | | AACAGAGGAGACAUAACAUG |
| | | | AACAAUCACAUACAAAGCAA |
| | | | ACGA (SEQ ID NO: 90) |

| NAND | |
|---|---|
| Inputs | |
| Input | |
| RNA | |
| Letter | Input RNA Sequence |
| A | GGGACAAGAACAAUACGGUUCUUGUACUAUCUA |
| | AUCUUACUAAAC (SEQ ID NO: 94) |
| B | GGGACAUAAACAUAGAGGUUUAUGUGAGUAGAA |
| | (UUGAUUGAUAAG (SEQ ID NO: 95) |
| C | GGGACAUACAAUGAACUUUGUAUGUGAUUUGUA |
| | UUUGAUUGUAGC (SEQ ID NO: 96) |
| D | GGGACCUAACAUAAACUUGUUAGGUGCGUAGAU |
| | CUGAUUAGUGUG (SEQ ID NO: 97) |
| E | GGGCGACACUAAUAGUGUAGUGUCGGAUAAGUG |
| | AAUAGUAGUAGA (SEQ ID NO: 98) |
| F | GGGACACUAACCAUAUAGUUAGUGUGCGUAAAG |
| | (UGAUAGGAGUAA SEQ ID NO: 99) |
| Noncognate | GGGCGAUAAUGUUCCGUCAUUAUCGGCAUUCUU |
| trigger | AUUCAAUCAUAG(SEQ ID NO: 100) |

TABLE 6
4-Input NAND Constructs

| | | Input RNAs | |
|---|---|---|---|
| | | Used | |
| | | (Listed by | |
| | | binding | |
| | | location | |
| | | 5' to 3' | |
| Device | Device | on switch | Switch RNA |
| Number | Name | RNA) | Sequence |
| 1 | STUR_NAND_ | F, D, C, B | GGGCUCCUAUCACU |
| | link_11_ | | UUACUUGUUAUAGU |
| | N04_010_ | | UAUGAACAGAGGAG |
| | FDCB | | ACAUAACAUGAACA |
| | | | AGCACACUAACUAC |
| | | | AAAUUCCAAAGCCA |
| | | | CAUACUAAUCAGAU |
| | | | CUACUUGUUAUAGU |
| | | | UAUGAACAGAGGAG |
| | | | ACAUAACAUGAACA |
| | | | AGCACCUAACAAGA |
| | | | CUAAUCCAAAUCAA |
| | | | AUCACAAUCAAAUA |
| | | | CAAAUUGUUAUAGU |
| | | | UAUGAACAGAGGAG |
| | | | ACAUAACAUGAACA |
| | | | AUCACAUACAAAGC |
| | | | AAACGACACAUACC |
| | | | UUCAUCAAUCAAUU |
| | | | CUACUUGUUAUAGU |
| | | | UAUGAACAGAGGAG |
| | | | ACAUAACAUGAACA |
| | | | AUCACAUAAACCUA |
| | | | AGAACUAACCUGGC |
| | | | GGCAGCGCAA |
| | | | (SEQ ID NO: 91) |
| 2 | STUR_NAND_ | F, B, D, C | GGGCUCCUAUCACU |
| | link17_ | | UUACUUGUUAUAGU |
| | N04_011_ | | UAUGAACAGAGGAG |
| | FBDC | | ACAUAACAUGAACA |
| | | | AGCACACUAACUAC |
| | | | AAAUUCCUAACUUU |
| | | | CAAUCCAGCAUCAA |
| | | | UCAAUUCUACUUGU |
| | | | UAUAGUUAUGAACA |
| | | | GAGGAGACAUAACA |
| | | | UGAACAAUCACAUA |
| | | | AACCUAAGAACUAA |
| | | | GCCGACAAUACUAA |
| | | | CACUAAUCAGAUCU |
| | | | ACUUGUUAUAGUUA |
| | | | UGAACAGAGGAGAC |
| | | | AUAACAUGAACAAG |
| | | | CACCUAACAAGACU |
| | | | AAUCAACUCAAUAU |
| | | | CGCAGAUACAAUCA |
| | | | AAUACAAAUUGUUA |
| | | | UAGUUAUGAACAGA |
| | | | GGAGACAUAACAUG |
| | | | AACAAUCACAUACA |
| | | | AAGCAAACGAAACC |
| | | | UGGCGGCAGCGCAA |
| | | | (SEQ ID NO: 92) |
| 3 | STUR_NAND_ | D, F, C, B | GGGACUAAUCAGAU |
| | link17_ | | CUACUUGUUAUAGU |
| | N04_001_ | | UAUGAACAGAGGAG |
| | DFCB | | ACAUAACAUGAACA |
| | | | AGCACCUAACAAGA |
| | | | CUAAUCAACUACAA |
| | | | AUACAAUAACUCCU |
| | | | AUCACUUUACUUGU |
| | | | UAUAGUUAUGAACA |
| | | | GAGGAGACAUAACA |
| | | | UGAACAAGCACACU |
| | | | AACUACAAAUUCAC |
| | | | AAUUACCGACGCAA |
| | | | UACAAUCAAAUACA |
| | | | AAUUGUUAUAGUUA |
| | | | UGAACAGAGGAGAC |
| | | | AUAACAUGAACAAU |
| | | | CACAUACAAAGCAA |
| | | | ACGAAAUACCGCAA |
| | | | AUACCAUAUCAAUC |
| | | | AAUUCUACUUGUUA |
| | | | UAGUUAUGAACAGA |
| | | | GGAGACAUAACAUG |
| | | | AACAAUCACAUAAA |
| | | | CCUAAGAACUAACC |
| | | | UGGCGGCAGCGCAA |
| | | | (SEQ ID NO: 93) |

### DISCUSSION

We have demonstrated a new type of de novo-designed translational repressor that exploits an unstable RNA secondary structure to enable signal output in the device ON state by facilitating ribosomal access and low OFF state expression by facilitating binding of a cognate trigger RNA with nearly arbitrary sequence. These NeaTTR systems provided remarkably high repression with ON/OFF ratios up to nearly 800-fold. They exhibited orthogonality that surpassed those reported for previous RNA-based repressors, forming an orthogonal library of 15 different devices expected to provide ON/OFF levels of at least 40-fold. We attribute the high repression levels of the devices to the favorable reaction kinetics and thermodynamics provided by precisely designed RNAs and the use of the near-threshold switch RNA structure to simultaneous enable translation and a strong binding site for interactions with the trigger RNA. A crucial part of the observed orthogonality of the NeaTTRs is the use of trigger RNAs in which a substantial portion of their interaction region with the switch RNA is sequestered within a hairpin. This design feature means that each trigger only provides 17 exposed nucleotides. Exposed, single-stranded trigger domains are a major source of device crosstalk in other repressors, and decrease output of noncognate switch RNAs by binding non-specifically and impairing RBS access. Since trigger RNAs are typically expressed at higher concentrations than the switch RNAs in vivo, these nonspecific damping effects can be substantial. Accordingly, reduction of the single-stranded region of the trigger RNA to only 17-nts substantially attenuates this effect. Lastly, our validation of the NeaTTRs in both in vivo contexts in E. coli and in vitro conditions in cell-free reactions means that they can be deployed in applications such as metabolic engineering, whole-cell biosensing, or for in vitro diagnostics.

Given the high dynamic range and orthogonality of these devices, it is expected that they can be deployed to regulate multiple enzymes within a metabolic pathway and enable fine control of enzyme levels through tuning of the levels of the corresponding trigger RNA. Moreover, entire metabolic pathways can be regulated by placing each enzyme under control of NeaTTRs that respond to the same trigger RNA. The ability of NeaTTRs to detect nearly arbitrary RNAs also means that they could be used to respond to the expression of endogenous or pathogen associated RNAs within the cell to provide dynamic and response regulation of gene expression. Such capabilities could be useful not only for metabolic engineering, but also for developing whole-cell biosensors or potentially for rapid, high-throughput screening antimicrobial compounds. For diagnostic applications, riboregulators that turn off gene expression in response a target RNA could be used prevent a given reaction from occurring, which could be helpful given the limited energy bandwidth of cell-free reactions, or to provide dynamic expression conditions, for instance the expression of a protein for a limited amount of time at the outset of a diagnostic test.

### RNA-Only Logic Systems:

The high dynamic range and orthogonality of NeaTTRs enable them to be incorporated into biomolecular circuits for evaluating complex logic expressions. The NeaTTRs on their own act as inverters with expression of the trigger RNA or input RNA turning off protein expression. In addition, NeaTTRs can be used to evaluate both NAND and NOR logic expressions. We show in FIG. 6 two different designs for carrying out NAND logic. Type I NAND logic can be achieved using an extended gate RNA that features multiple MeTR switch RNA modules concatenated upstream of an open reading frame (FIG. 6a). Each of the corresponding trigger RNAs for the switch modules thus functions as an input molecule. Expression of these input RNAs enables them to bind to the cognate sensor binding site and deactivate the RBS and start codon of the switch RNA. However, if one or more of the set of input RNAs is absent, the RBS and start codons of the corresponding switch RNAs will remain exposed and enable translation to still occur. On the other hand, if all the input RNAs are expressed, each of the switch RNAs will be repressed and translation will cease. This behavior corresponds to NAND logic. In order for such NAND gate RNAs to work effectively, care must be taken to ensure that none of the switch RNA modules nor the linker sequences that separate them encodes an in-frame stop codon. Furthermore, each of the switch RNAs must also be in the same reading frame as the downstream gene.

FIG. 6B shows the design of a Type II NAND circuit. This RNA circuit employs only a single switch RNA module but now divides the trigger RNA sequence between two different input RNAs 1 and 2 that have complementary hybridization domains. Upon expression of both inputs, the two will hybridize to one another and form a complete trigger RNA to abolish translation from the complementary switch RNA. When only one of the input RNAs is expressed, the region complementary to the switch RNA is designed to be sufficiently short to either prevent input RNA binding or to provide insufficient base pairing to prevent translation from occurring. Thus, gene expression is only turned off when both input RNAs are present, which corresponds to 2-input NAND logic. Like the Type I devices, Type II NAND logic can be extended to greater than 2-input expressions. For an N-input NAND circuit, the trigger RNA sequence can be divided among N different input RNAs that must hybridize to one another to generate a fully assembled and functional trigger RNA for the corresponding switch RNA. Alternatively, the trigger RNA can be divided between input bits 1 and N and the intervening bits 2 through N−1 can be designed to hybridize directly with another and form an N-RNA complex that can repress the switch RNA. In this design, input 2 would bind through two hybridization domains to input 1 and input 3. Or in general, input m would bind to inputs m−1 and m+1 through hybridization domains. Finally, a combination of both the above input RNA design strategies could be employed and spacers between hybridization domains and/or trigger RNA portions could be used to increase the activity of the final assembled trigger.

NOR logic requires RNA circuit output to be active only if all input RNAs are not expressed. The design of such a NOR gate circuit is shown in FIG. 7. This gate RNA features a single switch RNA located just upstream of the open reading from of the output gene. Upstream of the switch RNA are two or more hairpins each designed to detect the input RNA molecules of the circuit. Each of these hairpins contains the sequence of the cognate trigger for the switch RNA. However, these trigger sequences are sequestered within the stem loops of each of the hairpins. Consequently, in the absence of the corresponding input RNA, the trigger sequences are unable to hybridize to the sensor RNA recognition site and translation will continue to occur. When an input RNA is expressed, the input RNA will bind through a toehold-mediated linear-linear interaction and unwind most (or all depending on the final design) of the hairpin stem. The resulting weakened hairpin structure is no longer strongly sequestered and thus is available for binding to the switch RNA. Accordingly, the gate RNA will undergo an intramolecular refolding process and the newly exposed trigger RNA will bind to the switch RNA region. Following trigger binding, the RBS and start codon of the switch RNA will be strongly repressed and thus prevent translation from occurring. Since all of the upstream hairpins are designed to bind to an input RNA and harbor trigger RNA sites, binding of any input RNA to the gate RNA will cause output gene expression to be turned off. In principle, any number of input RNA sensing hairpins can be arrayed upstream of the switch RNA meaning that the system is capable of N-input NOR expressions. Lastly, the trigger RNA sequence need not comprise part of the stem of the input-sensing hairpin. The trigger RNA can be contained entirely within the loop of the hairpin, provided it is not sufficiently exposed to enable hybridization with the switch RNA sensing domain.

Importantly, this NOR gate concept can also be applied to other riboregulators beyond NeaTTRs. The upstream hairpins that sense the input RNAs can be designed to expose arbitrary RNA sequences. Thus, they can be used to activate (or deactivate) any existing riboregulator, including toehold switches⁶, toehold repressors, beacon riboregulators, and STARS⁹, among others. For repressing riboregulators, these types of gate RNAs would carry out NOR logic. For activating riboregulators like the toehold switch, these types of gate RNAs would carry out OR logic. An important advantage of this approach compared to OR logic gates of concatenated activating riboregulators, which are equivalent to the Type I NAND gate RNAs, is that translation of the output gene always begins at the same location and thus the output of the gate RNA is the same regardless of the input RNA used. Furthermore, gate RNAs of this type do not need to be screened for in-frame stop codons upstream of the start codon site, and translation will not be affected by the procession of the ribosome through areas in the gate RNA with high secondary structure, which occurs for gate RNAs of the Type I NAND variety. It is possible to implement this OR and NOR logic functionality of the gate RNA using separate RNA hairpin species that are not covalently bound to one another. However, the use of a single RNA to encode all the sensing hairpins leads to enhanced reaction co-localization, since both switch and trigger are within the same molecule and need not find each other in the cytoplasm or solution through random diffusion. Consequently, these extended gate RNAs should provide much better reaction kinetics and improved performance overall compared to less novel nucleic acid networks comprised of multiple distinct RNA molecules in place of the single gate RNA molecule.

### Materials and Experimental Procedures

Materials and Bacterial Strains: All DNA oligonucleotides were designed using NUPACK software package⁸ and purchased from Integrated DNA Technologies. E. coli strains DH5α (endA1 recA1 gyrA96 thi-1 glnV44 relA1 hsdR17(rK− mK+) λ−) and BL21 star DE3 (F-ompT hsdSB (rB−mB−) gal dcm rne131 [DE3]; Invitrogen) were used in this study.

Plasmid Construction: Both insert and vector backbone DNA oligonucleotides were amplified via PCR and assembled using Gibson assembly with 30-bp overlap regions¹². Triggers were inserted into pET15b-derived vectors, whereas the switch system was inserted into a pCOLADuet-derived vector.

Growth and Expression Conditions: E. coli were grown in LB broth with appropriate antibiotics at 37° C. For flow cytometry measurements, overnight cultures of cells picked from individual colonies were diluted 100 fold with fresh media shaken at 37° C. for 80 minutes before induction. 0.1 mM isopropyl β-D-1-thiogalactopyranoside (IPTG) were used to induce the cells. Measurements were taken at least 3 hours after induction.

Flow Cytometry Measurements and Analysis: Flow cytometry was performed using a Stratedigm S1000EXi flow cytometer with an A600 HTAS (High Throughput Auto Sampler). Cells were diluted with phosphate buffered saline (PBS) before measurement. Forward scatter (FSC) was used for trigger, and ˜40,000 individual cells were analyzed using custom Matlab scripts.

Cell-free Test with Plate Reader: PURExpress® In Vitro Protein Synthesis Kit (NEB) was used for cell free tests with 100 ng switch plasmid and 400 ng trigger plasmid in 20 μl reaction system. Synergy H1 Microplate reader (Biotek) was used to monitor GFP synthesis kinetics in 4 hours.

We claim:

1. A method for altering expression of a protein, the method comprising:
providing a protein coding sequence operably linked to a near-threshold translational repressor having first and second trigger recognition sequences that are fully or partially complementary to a repressing trigger RNA; and
providing the repressing trigger RNA, whereby expression of the protein is altered according to the level of repressing trigger RNA provided.

2. The method of claim 1, wherein expression of two or more proteins are altered by providing two or more protein coding sequences, each operably linked to a near-threshold translational repressor comprising first and second trigger recognition sequences that are fully or partially complementary to a single repressing trigger RNA.

3. The method of claim 1, wherein the fold-change in expression of the protein is at least 25 fold.

4. The method of claim 1, wherein the fold-change in expression of the protein is at least 50 fold.

5. A method for altering expression of a protein in a cell, the method comprising:
introducing into a cell a protein coding sequence operably linked to a near-threshold translational repressor having first and second trigger recognition sequences that are fully or partially complementary to a repressing trigger RNA; and
introducing into a cell the repressing trigger RNA, whereby expression of the protein is altered according to the level of repressing trigger RNA introduced to the cell.

6. The method of claim 5, wherein expression of two or more proteins are altered by introducing into the cell two or more protein coding sequences, each operably linked to a near-threshold translational repressor comprising first and second trigger recognition sequences that are fully or partially complementary to a single repressing trigger RNA.

7. The method of claim 5, wherein the fold-change in expression of the protein is at least 25 fold.

8. The method of claim 5, wherein the fold-change in expression of the protein is at least 50 fold.


SEQUENCE LISTING (177 sequences)

SEQ ID NO: 1 - RNA, 94 nt, synthetic construct
  misc_feature 1..94: note = STUR_NN_gen1_N020
  source 1..94: mol_type = other RNA
gggatgaatgatatacacttgttatagttatgaacagaggagacataacatgaacaagcacgaattgactacactaaacctggcggcagcgcaa

SEQ ID NO: 2 - RNA, 94 nt, synthetic construct
  misc_feature 1..94: note = STUR_NN_gen1_N010
  source 1..94: mol_type = other RNA
gggtagtaagattagatattgttatagttatgaacagaggagacataacatgaacaagtacaagaaccgaaacgagaacctggcggcagcgcaa

SEQ ID NO: 3 - RNA, 94 nt, synthetic construct
  misc_feature 1..94: note = STUR_WW_gen1_N008
  source 1..94: mol_type = other RNA
gggtactcaagaattccattgttatagttatgaacagaggagacataacatgaacaatcacatactaacattactaaacctggcggcagcgcaa

SEQ ID NO: 4 - RNA, 94 nt, synthetic construct
  misc_feature 1..94: note = STUR_NN_gen1_N021
  source 1..94: mol_type = other RNA
gggactactattcacttattgttatagttatgaacagaggagacataacatgaacaatccgacactacaatttcgaaacctggcggcagcgcaa

SEQ ID NO: 5 - RNA, 94 nt, synthetic construct
  misc_feature 1..94: note = STUR_NN_gen1_N013
  source 1..94: mol_type = other RNA
gggacaatcaaatacaaattgttatagttatgaacagaggagacataacatgaacaatcacatacaaagcaaacgaaacctggcggcagcgcaa

SEQ ID NO: 6 - RNA, 94 nt, synthetic construct
  misc_feature 1..94: note = STUR_NN_gen1_N002
  source 1..94: mol_type = other RNA
gggatgatattgaattacttgttatagttatgaacagaggagacataacatgaacaagcacactacactaaacataaacctggcggcagcgcaa

SEQ ID NO: 7 - RNA, 94 nt, synthetic construct
  misc_feature 1..94: note = STUR_NN_gen1_N015
  source 1..94: mol_type = other RNA
gggtccaaatcttacttattgttatagttatgaacagaggagacataacatgaacaatcactacctcgaatacttaaacctggcggcagcgcaa

SEQ ID NO: 8 - RNA, 94 nt, synthetic construct
  misc_feature 1..94: note = STUR_NN_gen1_N023
  source 1..94: mol_type = other RNA
gggcaaatactccatatcttgttatagttatgaacagaggagacataacatgaacaagcacataaactcactgataaacctggcggcagcgcaa

SEQ ID NO: 9 - RNA, 94 nt, synthetic construct
  misc_feature 1..94: note = STUR_NN_gen1_N024
  source 1..94: mol_type = other RNA
gggctcctatcactttacttgttatagttatgaacagaggagacataacatgaacaagcacactaactacaaattcaacctggcggcagcgcaa

SEQ ID NO: 10 - RNA, 94 nt, synthetic construct
  misc_feature 1..94: note = STUR_NN_gen1_N003
  source 1..94: mol_type = other RNA
gggagtataagatagatattgttatagttatgaacagaggagacataacatgaacaagtacgaagcaacgaacataaacctggcggcagcgcaa

SEQ ID NO: 11 - RNA, 94 nt, synthetic construct
  misc_feature 1..94: note = STUR_NN_gen1_N019
  source 1..94: mol_type = other RNA
gggactaatcagatctacttgttatagttatgaacagaggagacataacatgaacaagcacctaacaagactaatcaacctggcggcagcgcaa

SEQ ID NO: 12 - RNA, 94 nt, synthetic construct
  misc_feature 1..94: note = STUR_NN_gen1_N012
  source 1..94: mol_type = other RNA
gggtgattgaataagaatttgttatagttatgaacagaggagacataacatgaacaagccgataatgacactaactaacctggcggcagcgcaa

SEQ ID NO: 13 - RNA, 94 nt, synthetic construct
  misc_feature 1..94: note = STUR_WW_gen1_N010
  source 1..94: mol_type = other RNA
gggcaaagtatccatcatttgttatagttatgaacagaggagacataacatgaacaatcacatactagaatctaagaacctggcggcagcgcaa

SEQ ID NO: 14 - RNA, 94 nt, synthetic construct
  misc_feature 1..94: note = STUR_NN_gen1_N007
  source 1..94: mol_type = other RNA
gggcaagatttagtagatttgttatagttatgaacagaggagacataacatgaacaatccgaacgaccgaaatgataacctggcggcagcgcaa

SEQ ID NO: 15 - RNA, 94 nt, synthetic construct
  misc_feature 1..94: note = STUR_NN_gen1_N001
  source 1..94: mol_type = other RNA
gggcaattagtactatccttgttatagttatgaacagaggagacataacatgaacaagtcacataaccgcaaactaaacctggcggcagcgcaa

SEQ ID NO: 16 - RNA, 94 nt, synthetic construct
  misc_feature 1..94: note = STUR_NN_gen1_N009
  source 1..94: mol_type = other RNA
gggcgaagatgatacaatttgttatagttatgaacagaggagacataacatgaacaagccacctcacactcaacataacctggcggcagcgcaa

SEQ ID NO: 17 - RNA, 94 nt, synthetic construct
  misc_feature 1..94: note = STUR_WW_gen1_N016
  source 1..94: mol_type = other RNA
gggatactttcaaacttattgttatagttatgaacagaggagacataacatgaacaatcacactaatcgactgaataacctggcggcagcgcaa

SEQ ID NO: 18 - RNA, 94 nt, synthetic construct
  misc_feature 1..94: note = STUR_WW_gen1_N020
  source 1..94: mol_type = other RNA
gggtaccttcaaattccattgttatagttatgaacagaggagacataacatgaacaatcacctcactaacattatcaacctggcggcagcgcaa

SEQ ID NO: 19 - RNA, 94 nt, synthetic construct
  misc_feature 1..94: note = STUR_NN_gen1_N011
  source 1..94: mol_type = other RNA
gggatcaatcaattctacttgttatagttatgaacagaggagacataacatgaacaatcacataaacctaagaactaacctggcggcagcgcaa

SEQ ID NO: 20 - RNA, 94 nt, synthetic construct
  misc_feature 1..94: note = STUR_WW_gen1_N018
  source 1..94: mol_type = other RNA
gggattcacttacaagatttgttatagttatgaacagaggagacataacatgaacaatcacatagaccatagaacgaacctggcggcagcgcaa

SEQ ID NO: 21 - RNA, 94 nt, synthetic construct
  misc_feature 1..94: note = STUR_WW_gen1_N012
  source 1..94: mol_type = other RNA
gggatgtgattactagatttgttatagttatgaacagaggagacataacatgaacaatccgaacaagcaatacaagaacctggcggcagcgcaa

SEQ ID NO: 22 - RNA, 94 nt, synthetic construct
  misc_feature 1..94: note = STUR_WW_gen1_N009
  source 1..94: mol_type = other RNA
gggtccatatacttcaatttgttatagttatgaacagaggagacataacatgaacaatcaacctaattctgagataaacctggcggcagcgcaa

SEQ ID NO: 23 - RNA, 94 nt, synthetic construct
  misc_feature 1..94: note = STUR_WW_gen1_N023
  source 1..94: mol_type = other RNA
gggcaattcctaatcctattgttatagttatgaacagaggagacataacatgaacaatccaaatagagcaaagcagaacctggcggcagcgcaa

SEQ ID NO: 24 - RNA, 94 nt, synthetic construct
  misc_feature 1..94: note = STUR_NN_gen1_N008
  source 1..94: mol_type = other RNA
gggacaaatcagataaacttgttatagttatgaacagaggagacataacatgaacaagccaatagatagaacacgaaacctggcggcagcgcaa

SEQ ID NO: 25 - RNA, 94 nt, synthetic construct
  misc_feature 1..94: note = STUR_WW_gen1_N011
  source 1..94: mol_type = other RNA
gggaatccaagttatcatttgttatagttatgaacagaggagacataacatgaacaatacacataaagacctcataaacctggcggcagcgcaa

SEQ ID NO: 26 - RNA, 94 nt, synthetic construct
  misc_feature 1..94: note = STUR_WW_gen1_N014
  source 1..94: mol_type = other RNA
gggatgatcgaaacgaatttgttatagttatgaacagaggagacataacatgaacaatacaagaatagacaatagaaacctggcggcagcgcaa

SEQ ID NO: 27 - RNA, 94 nt, synthetic construct
  misc_feature 1..94: note = STUR_WW_gen1_N003
  source 1..94: mol_type = other RNA
gggaatgaagatagtgatttgttatagttatgaacagaggagacataacatgaacaatccgaataagcgaactaagaacctggcggcagcgcaa

SEQ ID NO: 28 - RNA, 94 nt, synthetic construct
  misc_feature 1..94: note = STUR_WW_gen1_N017
  source 1..94: mol_type = other RNA
gggatctcaccgtgtctattgttatagttatgaacagaggagacataacatgaacaatcaacatacatcatacaagaacctggcggcagcgcaa

SEQ ID NO: 29 - RNA, 94 nt, synthetic construct
  misc_feature 1..94: note = STUR_WW_gen1_N005
  source 1..94: mol_type = other RNA
gggactccaattgacgatttgttatagttatgaacagaggagacataacatgaacaatacatatagactaaacataaacctggcggcagcgcaa

SEQ ID NO: 30 - RNA, 94 nt, synthetic construct
  misc_feature 1..94: note = STUR_NN_gen1_N004
  source 1..94: mol_type = other RNA
gggtaatgaagtgaatacttgttatagttatgaacagaggagacataacatgaacaagccaaatgatactaaaccgaacctggcggcagcgcaa

SEQ ID NO: 31 - RNA, 94 nt, synthetic construct
  misc_feature 1..94: note = STUR_NN_gen1_N016
  source 1..94: mol_type = other RNA
gggtcccattatcttaccttgttatagttatgaacagaggagacataacatgaacaagcctacactaccattcccgaacctggcggcagcgcaa

SEQ ID NO: 32 - RNA, 94 nt, synthetic construct
  misc_feature 1..94: note = STUR_WW_gen1_N006
  source 1..94: mol_type = other RNA
gggcgatatgagtaaagattgttatagttatgaacagaggagacataacatgaacaatgcaatagaacgatacaagaacctggcggcagcgcaa

SEQ ID NO: 33 - RNA, 94 nt, synthetic construct
  misc_feature 1..94: note = STUR_WW_gen1_N024
  source 1..94: mol_type = other RNA
gggactactccattctatttgttatagttatgaacagaggagacataacatgaacaatccatacctcatacaacgaaacctggcggcagcgcaa

SEQ ID NO: 34 - RNA, 94 nt, synthetic construct
  misc_feature 1..94: note = STUR_WW_gen1_N021
  source 1..94: mol_type = other RNA
gggcgacttaaactactattgttatagttatgaacagaggagacataacatgaacaatcactccacggaaactactaacctggcggcagcgcaa

SEQ ID NO: 35 - RNA, 94 nt, synthetic construct
  misc_feature 1..94: note = STUR_WW_gen1_N013
  source 1..94: mol_type = other RNA
gggatgtgaacttaaagattgttatagttatgaacagaggagacataacatgaacaatgcgaaatacggaaatagaaacctggcggcagcgcaa

SEQ ID NO: 36 - RNA, 94 nt, synthetic construct
  misc_feature 1..94: note = STUR_NN_gen1_N018
  source 1..94: mol_type = other RNA
gggcaatcctattcatcattgttatagttatgaacagaggagacataacatgaacaattctcaacatcaatcactcaacctggcggcagcgcaa

SEQ ID NO: 37 - RNA, 94 nt, synthetic construct
  misc_feature 1..94: note = STUR_WW_gen1_N001
  source 1..94: mol_type = other RNA
gggtaagaattagaatcattgttatagttatgaacagaggagacataacatgaacaatcagcgaacacgaaacataaacctggcggcagcgcaa

SEQ ID NO: 38 - RNA, 94 nt, synthetic construct
  misc_feature 1..94: note = STUR_NN_gen1_N022
  source 1..94: mol_type = other RNA
gggtcaatccgataatctttgttatagttatgaacagaggagacataacatgaacaattactccaaggcaatgataaacctggcggcagcgcaa

SEQ ID NO: 39 - RNA, 94 nt, synthetic construct
  misc_feature 1..94: note = STUR_WW_gen1_N007
  source 1..94: mol_type = other RNA
gggaatgaatgatgagatttgttatagttatgaacagaggagacataacatgaacaatgagcgaacgagcaatggaaacctggcggcagcgcaa

SEQ ID NO: 40 - RNA, 94 nt, synthetic construct
  misc_feature 1..94: note = STUR_NN_gen1_N006
  source 1..94: mol_type = other RNA
gggatttagaagtaagtattgttatagttatgaacagaggagacataacatgaacaattcgaagatatgggacgataacctggcggcagcgcaa

SEQ ID NO: 41 - RNA, 94 nt, synthetic construct
  misc_feature 1..94: note = STUR_WW_gen1_N004
  source 1..94: mol_type = other RNA
gggatttacttagatactttgttatagttatgaacagaggagacataacatgaacaattacatatacatacgaacgaacctggcggcagcgcaa

SEQ ID NO: 42 - RNA, 94 nt, synthetic construct
  misc_feature 1..94: note = STUR_WW_gen1_N015
  source 1..94: mol_type = other RNA
gggcgaattgaaatgaaattgttatagttatgaacagaggagacataacatgaacaatgacggaatagaaactaagaacctggcggcagcgcaa

SEQ ID NO: 43 - RNA, 94 nt, synthetic construct
  misc_feature 1..94: note = STUR_WW_gen1_N019
  source 1..94: mol_type = other RNA
gggcatttaccctactatttgttatagttatgaacagaggagacataacatgaacaatacactccaaatcaaagataacctggcggcagcgcaa

SEQ ID NO: 44 - RNA, 94 nt, synthetic construct
  misc_feature 1..94: note = STUR_WW_gen1_N002
  source 1..94: mol_type = other RNA
gggtattacttaccgatattgttatagttatgaacagaggagacataacatgaacaatcactccaacggaacaatcaacctggcggcagcgcaa

SEQ ID NO: 45 - RNA, 94 nt, synthetic construct
  misc_feature 1..94: note = STUR_WW_gen1_N022
  source 1..94: mol_type = other RNA
gggctcatacttcactatttgttatagttatgaacagaggagacataacatgaacaatacacctaacataatcactaacctggcggcagcgcaa

SEQ ID NO: 46 - RNA, 94 nt, synthetic construct
  misc_feature 1..94: note = STUR_NN_gen1_N005
  source 1..94: mol_type = other RNA
gggatctccatctatccattgttatagttatgaacagaggagacataacatgaacaatccattaagtaacaagcagaacctggcggcagcgcaa

SEQ ID NO: 47 - RNA, 94 nt, synthetic construct
  misc_feature 1..94: note = STUR_NN_gen1_N014
  source 1..94: mol_type = other RNA
gggtagattaagagtgatttgttatagttatgaacagaggagacataacatgaacaatgacctaaacgcacgaactaacctggcggcagcgcaa

SEQ ID NO: 48 - RNA, 94 nt, synthetic construct
  misc_feature 1..94: note = STUR_NN_gen1_N017
  source 1..94: mol_type = other RNA
gggcaatctccaatacgtttgttatagttatgaacagaggagacataacatgaacaagcacttaaaccgaataactaacctggcggcagcgcaa

SEQ ID NO: 49 - RNA, 203 nt, synthetic construct
  misc_feature 1..203: note = STUR_NOR_Gen3_Hpin5
  source 1..203: mol_type = other RNA
gggatcaaagtatacaagggtagggtagggttatcgcgacactaatagtgtagtgtcggataagtgaatagtagtcgataaccctaccctaccgcctcaattcgaggcaagactactattcacttattgttatagttatgaacagaggagacataacatgaacaatccgacactacaatttcgaaacctggcggcagcgcaaa

SEQ ID NO: 50 - RNA, 203 nt, synthetic construct
  misc_feature 1..203: note = STUR_NOR_Gen2_Hpin1
  source 1..203: mol_type = other RNA
gggtcttatgtgtatgtttctgtcttgtctttatctcgacactaatagtgtagtgtcggataagtgaatagtagtagataaagacaagacagagcctcaattcgaggcaagactactattcacttattgttatagttatgaacagaggagacataacatgaacaatccgacactacaatttcgaaacctggcggcagcgcaaa

SEQ ID NO: 51 - RNA, 202 nt, synthetic construct
  misc_feature 1..202: note = STUR_NOR_Gen3_Hpin7_mut
  source 1..202: mol_type = other RNA
gggagtaaagtatagaacgcttgatgttgatgttagcgacactaatagtgtagtgtcggataagtgaatatagtctaacatcaacatcaagcgcctcaattcgaggcaagactactattcacttattgttatagttatgaacagaggagacataacatgaacaatccgacactacaatttcgaaacctggcggcagcgcaaa

SEQ ID NO: 52 - RNA, 202 nt, synthetic construct
  misc_feature 1..202: note = STUR_NOR_Gen2_Hpin9_mutRepstem
  source 1..202: mol_type = other RNA
gggtcgtataagtttaagtctgtttctgtctgttctcgacactaatagtgtagtgtcggataagtgaatagtagtagaacagacagaaacagagcctcaattcgaggcaagactactattcacttattgttaagttatgaacagaggagacataacatgaacaatccgacactacaatttcgaaacctggcggcagcgcaaa

SEQ ID NO: 53 - RNA, 203 nt, synthetic construct
  misc_feature 1..203: note = STUR_NOR_Gen3_Hpin11
  source 1..203: mol_type = other RNA
gggactaagactaaatgatgtatagattgaagattgcgacactaatagtgtagtgtcggataagtgaatagtagtcaatcttcaatctatacagcctcaattcgaggcaagactactattcacttattgttatagttatgaacagaggagacataacatgaacaatccgacactacaatttcgaaacctggcggcagcgcaaa

SEQ ID NO: 54 - RNA, 203 nt, synthetic construct
  misc_feature 1..203: note = STUR_NOR_Gen2_Hpin13
  source 1..203: mol_type = other RNA
gggttcttagtttatgtaggtctgttatcttgttctcgacactaatagtgtagtgtcggataagtgaatagtagtagaacaagataacagaccgcctcaattcgaggcaagactactattcacttattgttatagttatgaacagaggagacataacatgaacaatccgacactacaatttcgaaacctggcggcagcgcaaa

SEQ ID NO: 55 - RNA, 202 nt, synthetic construct
  misc_feature 1..202: note = STUR_NOR_Gen3_Hpin6_mut
  source 1..202: mol_type = other RNA
gggacgacttaagattacggtatgagtttagatttgcgacactaatagtgtagtgtcggataagtaatagtagtcaaatctaaactcataccgcctcaattcgaggcaagactactattcacttattgttatagttatgaacagaggagacataacatgaacaatccgacactacaatttcgaaacctggcggcagcgcaaa

SEQ ID NO: 56 - RNA, 203 nt, synthetic construct
  misc_feature 1..203: note = STUR_NOR_Gen2_Hpin12
  source 1..203: mol_type = other RNA
gggttaggtatgtgtattgattgatttgatgcttgtcgacactaatagtgtagtgtcggataagtgaatagtagtacaagcatcaaatcaatcgcctcaattcgaggcaagactactattcacttattgttatagttatgaacagaggagacataacatgaacaatccgacactacaatttcgaaacctggcggcagcgcaaa

SEQ ID NO: 57 - RNA, 203 nt, synthetic construct
  misc_feature 1..203: note = STUR_NOR_Gen2_Hpin16
  source 1..203: mol_type = other RNA
gggagtaaggtaaggtgagtgcgttgtctttagtctcgacactaatagtgtagtgtcggataagtgaatagtagtagactaaagacaacgcacgcctcaattcgaggcaagactactattcacttattgttatagttatgaacagaggagacataacatgaacaatccgacactacaatttcgaaacctggcggcagcgcaaa

SEQ ID NO: 58 - RNA, 203 nt, synthetic construct
  misc_feature 1..203: note = STUR_NOR_Gen2_Hpin3
  source 1..203: mol_type = other RNA
gggtctgtttatgtgtatgtgtctgtcttgtctgctcgacactaatagtgtagtgtcggataagtgaatagtagtagcagacaagacagacacgcctcaattcgaggcaagactactattcacttattgttatagttatgaacagaggagacataacatgaacaatccgacactacaatttcgaaacctggcggcagcgcaaa

SEQ ID NO: 59 - RNA, 203 nt, synthetic construct
  misc_feature 1..203: note = STUR_NOR_Gen3_Hpin3_mut
  source 1..203: mol_type = other RNA
gggagcttaatgatagaatgcgttgatgttagtaagcgacactaatagtgtagtgtcggataagtgaatagtagtcttactaacatcaacgcagcctcaattcgaggcaagactactattcacttattgttatagttatgaacagaggagacataacatgaacaatccgacactacaatttcgaaacctggcggcagcgcaaa

SEQ ID NO: 60 - RNA, 203 nt, synthetic construct
  misc_feature 1..203: note = STUR_NOR_Gen3_Hpin7
  source 1..203: mol_type = other RNA
gggagtaaagtatagaacgcttgatgttgatgttagcgacactaatagtgtagtgtcggataagtgaatagtagtctaacatcaacatcaagcgcctcaattcgaggcaagactactattcacttattgttatagttatgaacagaggagacataacatgaacaatccgacactacaatttcgaaacctggcggcagcgcaaa

SEQ ID NO: 61 - RNA, 203 nt, synthetic construct
  misc_feature 1..203: note = STUR_NOR_Gen2_Hpin2
  source 1..203: mol_type = other RNA
gggagttgtatgtgtatggtctgtcttgttctatctcgacactaatagtgtagtgtcggataagtgaatagtagtagatagaacaagacagacgcctcaattcgaggcaagactactattcacttattgttatagttatgaacagaggagacataacatgaacaatccgacactacaatttcgaaacctggcggcagcgcaaa

SEQ ID NO: 62 - RNA, 202 nt, synthetic construct
  misc_feature 1..202: note = STUR_NOR_Gen3_Hpin4_mut
  source 1..202: mol_type = other RNA
gggatgataaagattgatgctgttatgttgatttagcgacactaatagtgtagtgtcggataaggaatagtagtctaaatcaacataacagcgcctcaattcgaggcaagactactattcacttattgttatagttatgaacagaggagacataacatgaacaatccgacactacaatttcgaaacctggcggcagcgcaaa

SEQ ID NO: 63 - RNA, 202 nt, synthetic construct
  misc_feature 1..202: note = STUR_NOR_Gen3_Hpin4_mut
  source 1..202: mol_type = other RNA
gggatgataaagattgatgctgttatgttgatttagcgacactaatagtgtagtgtcggataatgaatagtagtctaaatcaacataacagcgcctcaattcgaggcaagactactattcacttattgttatagttatgaacagaggagacataacatgaacaatccgacactacaatttcgaaacctggcggcagcgcaaa

SEQ ID NO: 64 - RNA, 203 nt, synthetic construct
  misc_feature 1..203: note = STUR_NOR_Gen2_Hpin6
  source 1..203: mol_type = other RNA
gggagtaaagtaaaggtgtcgttgtatgttcgttctcgacactaatagtgtagtgtcggataagtgaatagtagtagaacgaacatacaacgagcctcaattcgaggcaagactactattcacttattgttatagttatgaacagaggagacataacatgaacaatccgacactacaatttcgaaacctggcggcagcgcaaa

SEQ ID NO: 65 - RNA, 203 nt, synthetic construct
  misc_feature 1..203: note = STUR_NOR_Gen2_Hpin15
  source 1..203: mol_type = other RNA
gggtctagtgtcgtcgttgtcttgtatggttcttgtcgacactaatagtgtagtgtcggataagtgaatagtagtacaagaaccatacaagacgcctcaattcgaggcaagactactattcacttattgttatagttatgaacagaggagacataacatgaacaatccgacactacaatttcgaaacctggcggcagcgcaaa

SEQ ID NO: 66 - RNA, 203 nt, synthetic construct
  misc_feature 1..203: note = STUR_NOR_Gen2_Hpin18
  source 1..203: mol_type = other RNA
gggttagtattgtagttgtctgtatttgtctctgctcgacactaatagtgtagtgtcggataagtgaatagtagtagcagagacaaatacagagcctcaattcgaggcaagactactattcacttattgttatagttatgaacagaggagacataacatgaacaatccgacactacaatttcgaaacctggcggcagcgcaaa

SEQ ID NO: 67 - RNA, 202 nt, synthetic construct
  misc_feature 1..202: note = STUR_NOR_Gen3_Hpin5_mutA
  source 1..202: mol_type = other RNA
gggatcaaagtatacaagggtagggtagggttatcgcgacactaatagtgtagtgtcggataagtgatagtagtcgataaccctaccctaccgcctcaattcgaggcaagactactattcacttattgttatagttatgaacagaggagacataacatgaacaatccgacactacaatttcgaaacctggcggcagcgcaaa

SEQ ID NO: 68 - RNA, 203 nt, synthetic construct
  misc_feature 1..203: note = STUR_NOR_Gen3_Hpin10_mut
  source 1..203: mol_type = other RNA
gggactggatacttatacggttctgtaggttcttagcgacactaatagtgtagtgtcggataagttaatagtagtctaagaacctacagaaccgcctcaattcgaggcaagactactattcacttattgttatagttatgaacagaggagacataacatgaacaatccgacactacaatttcgaaacctggcggcagcgcaaa

SEQ ID NO: 69 - RNA, 203 nt, synthetic construct
  misc_feature 1..203: note = STUR_NOR_Gen2_Hpin4
  source 1..203: mol_type = other RNA
gggttagtgtatggtatgtcgttcttgttcttatctcgacactaatagtgtagtgtcggataagtgaatagtagtagataagaacaagaacgagcctcaattcgaggcaagactactattcacttattgttatagttatgaacagaggagacataacatgaacaatccgacactacaatttcgaaacctggcggcagcgcaaa

SEQ ID NO: 70 - RNA, 203 nt, synthetic construct
  misc_feature 1..203: note = STUR_NOR_Gen3_Hpin8
  source 1..203: mol_type = other RNA
gggacgtactaattgatttcgtcttgtcttgtattgcgacactaatagtgtagtgtcggataagtgaatagtagtcaatacaagacaagacgagcctcaattcgaggcaagactactattcacttattgttatagttatgaacagaggagacataacatgaacaatccgacactacaatttcgaaacctggcggcagcgcaaa

SEQ ID NO: 71 - RNA, 203 nt, synthetic construct
  misc_feature 1..203: note = STUR_NOR_Gen2_Hpin5
  source 1..203: mol_type = other RNA
gggagtgtattgtatgtgtcttgttcttgttcgtctcgacactaatagtgtagtgtcggataagtgaatagtagtagacgaacaagaacaagagcctcaattcgaggcaagactactattcacttattgttatagttatgaacagaggagacataacatgaacaatccgacactacaatttcgaaacctggcggcagcgcaaa

SEQ ID NO: 72 - RNA, 203 nt, synthetic construct
  misc_feature 1..203: note = STUR_NOR_Gen2_Hpin14
  source 1..203: mol_type = other RNA
gggtcctgtgtctttatgtcttgttctgtttatcttcgacactaatagtgtagtgtcggataagtgaatagtagtaagataaacagaacaagagcctcaattcgaggcaagactactattcacttattgttatagttatgaacagaggagacataacatgaacaatccgacactacaatttcgaaacctggcggcagcgcaaa

SEQ ID NO: 73 - RNA, 202 nt, synthetic construct
  misc_feature 1..202: note = STUR_NOR_Gen2_Hpin17_mut
  source 1..202: mol_type = other RNA
gggtctgtgtatttctgttcttatgtcttgtcttatcgacactaatagtgtagtgtcggataagtgaaagtagtataagacaagacataagagcctcaattcgaggcaagactactattcacttattgttatagttatgaacagaggagacataacatgaacaatccgacactacaatttcgaaacctggcggcagcgcaaa

SEQ ID NO: 74 - RNA, 203 nt, synthetic construct
  misc_feature 1..203: note = STUR_NOR_Gen2_Hpin10
  source 1..203: mol_type = other RNA
gggatgtatgtgtaatggtctgtttatgtatgttctcgacactaatagtgtagtgtcggataagtgaatagtagtagaacatacataaacagagcctcaattcgaggcaagactactattcacttattgttatagttatgaacagaggagacataacatgaacaatccgacactacaatttcgaaacctggcggcagcgcaaa

SEQ ID NO: 75 - RNA, 202 nt, synthetic construct
  misc_feature 1..202: note = STUR_NOR_Gen3_Hpin2
  source 1..202: mol_type = other RNA
ggaatcttaatcgtataggtctttgtcttgttctgcgacactaatagtgtagtgtcggataagtgaatagtagtcagaacaagacaaagaccgcctcaattcgaggcaagactactattcacttattgttatagttatgaacagaggagacataacatgaacaatccgacactacaatttcgaaacctggcggcagcgcaaa

SEQ ID NO: 76 - RNA, 203 nt, synthetic construct
  misc_feature 1..203: note = STUR_NOR_Gen3_Hpin9
  source 1..203: mol_type = other RNA
gggagtagtagtaaatgtgtcttgtatcttgttctgcgacactaatagtgtagtgtcggataagtgaatagtagtcagaacaagatacaagacgcctcaattcgaggcaagactactattcacttattgttatagttatgaacagaggagacataacatgaacaatccgacactacaatttcgaaacctggcggcagcgcaaa

SEQ ID NO: 77 - RNA, 203 nt, synthetic construct
  misc_feature 1..203: note = STUR_NOR_Gen2_Hpin11
  source 1..203: mol_type = other RNA
gggagtaagttgaaagtaggttatgtttatgttgctcgacactaatagtgtagtgtcggataagtgaatagtagtagcaacataaacataaccgcctcaattcgaggcaagactactattcacttattgttatagttatgaacagaggagacataacatgaacaatccgacactacaatttcgaaacctggcggcagcgcaaa

SEQ ID NO: 78 - RNA, 292 nt, synthetic construct
  misc_feature 1..292: note = 2-input Switch RNA Sequence
  source 1..292: mol_type = other RNA
gggtcgtataagtttaagtctgtttctgtctgttctcgacactaatagtgtagtgtcggataagtgaatagtagtagaacagacagaaacagagcctcaattcgaggcaagtcttatgtgtatgtttctgtcttgtctttatctcgacactaatagtgtagtgtcggataagtgaatagtagtagataaagacaagacagagcctcaattcgaggcaagactactattcacttattgttatagttatgaacagaggagacataacatgaacaatccgacactacaatttcga

SEQ ID NO: 79 - RNA, 62 nt, synthetic construct
  misc_feature 1..62: note = Input A
  source 1..62: mol_type = other RNA
gggacctcaattcatctgggaattgaggtagaacagacagaaacagacttaaacttatacga

SEQ ID NO: 80 - RNA, 62 nt, synthetic construct
  misc_feature 1..62: note = Input B
  source 1..62: mol_type = other RNA
gggacaaggactctgaagtgagtccttgtagataaagacaagacagaaacatacacataaga

SEQ ID NO: 81 - RNA, 160 nt, synthetic construct
  misc_feature 1..160: note = STUR_NAND_L11_N008_HpFD
  source 1..160: mol_type = other RNA
gggctcctatcactttacttgttatagttatgaacagaggagacataacatgaacaagcacactaactacaaattccaaagccacatactaatcagatctacttgttatagttatgaacagaggagacataacatgaacaagcacctaacaagactaatc

SEQ ID NO: 82 - RNA, 166 nt, synthetic construct
  misc_feature 1..166: note = STUR_NAND_L17_N006_HpFD
  source 1..166: mol_type = other RNA
gggctcctatcactttacttgttatagttatgaacagaggagacataacatgaacaagcacactaactacaaattcaccgcaaatacaataacactaatcagatctacttgttatagttatgaacagaggagacataacatgaacaagcacctaacaagactaatc

SEQ ID NO: 83 - RNA, 166 nt, synthetic construct
  misc_feature 1..166: note = STUR_NAND_L17_N008_HpDB
  source 1..166: mol_type = other RNA
gggactaatcagatctacttgttatagttatgaacagaggagacataacatgaacaagcacctaacaagactaatcatatacaatacctccaaatcaatcaattctacttgttatagttatgaacagaggagacataacatgaacaatcacataaacctaagaact

SEQ ID NO: 84 - RNA, 160 nt, synthetic construct
  misc_feature 1..160: note = STUR_NAND_L11_N007_HpDC
  source 1..160: mol_type = other RNA
gggactaatcagatctacttgttatagttatgaacagaggagacataacatgaacaagcacctaacaagactaatccaaatcaaatcacaatcaaatacaaattgttatagttatgaacagaggagacataacatgaacaatcacatacaaagcaaacga

SEQ ID NO: 85 - RNA, 160 nt, synthetic construct
  misc_feature 1..160: note = STUR_NAND_L11_N002_HpCF
  source 1..160: mol_type = other RNA
gggacaatcaaatacaaattgttatagttatgaacagaggagacataacatgaacaatcacatacaaagcaaacgaacaatacccgactcctatcactttacttgttatagttatgaacagaggagacataacatgaacaagcacactaactacaaattc

SEQ ID NO: 86 - RNA, 166 nt, synthetic construct
  misc_feature 1..166: note = STUR_NAND_L17_N001_HpCD
  source 1..166: mol_type = other RNA
gggacaatcaaatacaaattgttatagttatgaacagaggagacataacatgaacaatcacatacaaagcaaacgaaacataaagccaactaaactaatcagatctacttgttatagttatgaacagaggagacataacatgaacaagcacctaacaagactaatc

SEQ ID NO: 87 - RNA, 256 nt, synthetic construct
  misc_feature 1..256: note = STUR_NAND_link17_N03_003_HpCBD
  source 1..256: mol_type = other RNA
gggacaatcaaatacaaattgttatagttatgaacagaggagacataacatgaacaatcacatacaaagcaaacgaaataccgcaaataccatatcaatcaattctacttgttatagttatgaacagaggagacataacatgaacaatcacataaacctaagaactaagccgacaatactaacactaatcagatctacttgttatagttatgaacagaggagacataacatgaacaagcacctaacaagactaatc

SEQ ID NO: 88 - RNA, 244 nt, synthetic construct
  misc_feature 1..244: note = STUR_NAND_link11_N03_004_HpBCD
  source 1..244: mol_type = other RNA
gggatcaatcaattctacttgttatagttatgaacagaggagacataacatgaacaatcacataaacctaagaactcaatatcgcatacaatcaaatacaaattgttatagttatgaacagaggagacataacatgaacaatcacatacaaagcaaacgacaaatacaaatactaatcagatctacttgttatagttatgaacagaggagacataacatgaacaagcacctaacaagactaatc

SEQ ID NO: 89 - RNA, 244 nt, synthetic construct
  misc_feature 1..244: note = STUR_NAND_link11_N03_006_HpFCD
  source 1..244: mol_type = other RNA
gggctcctatcactttacttgttatagttatgaacagaggagacataacatgaacaagcacactaactacaaattcgcaaatacatcacaatcaaatacaaattgttatagttatgaacagaggagacataacatgaacaatcacatacaaagcaaacgacaaatacaaatactaatcagatctacttgttatagttatgaacagaggagacataacatgaacaagcacctaacaagactaatc

SEQ ID NO: 90 - RNA, 244 nt, synthetic construct
  misc_feature 1..244: note = STUR_NAND_link11_N03_001_HpFDC
  source 1..244: mol_type = other RNA
gggctcctatcactttacttgttatagttatgaacagaggagacataacatgaacaagcacactaactacaaattccaaagccacatactaatcagatctacttgttatagttatgaacagaggagacataacatgaacaagcacctaacaagactaatccaaatcaaatcacaatcaaatacaaattgttatagttatgaacagaggagacataacatgaacaatcacatacaaagcaaacga

SEQ ID NO: 91 - RNA, 346 nt, synthetic construct
  misc_feature 1..346: note = STUR_NAND_link11_N04_010_FDCB
  source 1..346: mol_type = other RNA
gggctcctatcactttacttgttatagttatgaacagaggagacataacatgaacaagcacactaactacaaattccaaagccacatactaatcagatctacttgttatagttatgaacagaggagacataacatgaacaagcacctaacaagactaatccaaatcaaatcacaatcaaatacaaattgttatagttatgaacagaggagacataacatgaacaatcacatacaaagcaaacgacacataccttcatcaatcaattctacttgttatagttatgaacagaggagacataacatgaacaatcacataaacctaagaactaacctggcggcagcgcaa

SEQ ID NO: 92 - RNA, 364 nt, synthetic construct
  misc_feature 1..364: note = STUR_NAND_link17_N04_011_FBDC
  source 1..364: mol_type = other RNA
gggctcctatcactttacttgttatagttatgaacagaggagacataacatgaacaagcacactaactacaaattcctaactttcaatccagcatcaatcaattctacttgttatagttatgaacagaggagacataacatgaacaatcacataaacctaagaactaagccgacaatactaacactaatcagatctacttgttatagttatgaacagaggagacataacatgaacaagcacctaacaagactaatcaactcaatatcgcagatacaatcaaatacaaattgttatagttatgaacagaggagacataacatgaacaatcacatacaaagcaaacgaaacctggcggcagcgcaa

SEQ ID NO: 93 - RNA, 364 nt, synthetic construct
  misc_feature 1..364: note = STUR_NAND_link17_N04_001_DFCB
  source 1..364: mol_type = other RNA
gggactaatcagatctacttgttatagttatgaacagaggagacataacatgaacaagcacctaacaagactaatcaactacaaatacaataactcctatcactttacttgttatagttatgaacagaggagacataacatgaacaagcacactaactacaaattcacaattaccgacgcaatacaatcaaatacaaattgttatagttatgaacagaggagacataacatgaacaatcacatacaaagcaaacgaaataccgcaaataccatatcaatcaattctacttgttatagttatgaacagaggagacataacatgaacaatcacataaacctaagaactaacctggcggcagcgcaa

SEQ ID NO: 94 - RNA, 45 nt, synthetic construct
  misc_feature 1..45: note = synthetic
  source 1..45: mol_type = other RNA
gggacaagaacaatacggttcttgtactatctaatcttactaaac

SEQ ID NO: 95 - RNA, 45 nt, synthetic construct
  misc_feature 1..45: note = synthetic
  source 1..45: mol_type = other RNA
gggacataaacatagaggtttatgtgagtagaattgattgataag

SEQ ID NO: 96 - RNA, 45 nt, synthetic construct
  misc_feature 1..45: note = synthetic
  source 1..45: mol_type = other RNA
gggacatacaatgaactttgtatgtgatttgtatttgattgtagc

SEQ ID NO: 97 - RNA, 45 nt, synthetic construct
  misc_feature 1..45: note = synthetic
  source 1..45: mol_type = other RNA
gggacctaacataaacttgttaggtgcgtagatctgattagtgtg

SEQ ID NO: 98 - RNA, 45 nt, synthetic construct
  misc_feature 1..45: note = synthetic
  source 1..45: mol_type = other RNA
gggcgacactaatagtgtagtgtcggataagtgaatagtagtaga

SEQ ID NO: 99 - RNA, 45 nt, synthetic construct
  misc_feature 1..45: note = synthetic
  source 1..45: mol_type = other RNA
gggacactaaccatatagttagtgtgcgtaaagtgataggagtaa

SEQ ID NO: 100 - RNA, 45 nt, synthetic construct
  misc_feature 1..45: note = synthetic
  source 1..45: mol_type = other RNA
gggcgataatgttccgtcattatcggcattcttattcaatcatag

SEQ ID NO: 101 - RNA, 45 nt, synthetic construct
  misc_feature 1..45: note = synthetic
  source 1..45: mol_type = other RNA
gggacgaattgatttgtcaattcgtgcgtgtatatcattcatcat

SEQ ID NO: 102 - RNA, 45 nt, synthetic construct; identical to SEQ ID NO: 94
  misc_feature 1..45: note = synthetic
  source 1..45: mol_type = other RNA

SEQ ID NO: 103 - RNA, 45 nt, synthetic construct
  misc_feature 1..45: note = synthetic
  source 1..45: mol_type = other RNA
gggacatactatatcgttagtatgtgatggaattcttgagtaatg

SEQ ID NO: 104 - RNA, 45 nt, synthetic construct; identical to SEQ ID NO: 98
  misc_feature 1..45: note = synthetic
  source 1..45: mol_type = other RNA

SEQ ID NO: 105 - RNA, 45 nt, synthetic construct; identical to SEQ ID NO: 96
  misc_feature 1..45: note = synthetic
  source 1..45: mol_type = other RNA

SEQ ID NO: 106 - RNA, 45 nt, synthetic construct
  misc_feature 1..45: note = synthetic
  source 1..45: mol_type = other RNA
gggacactacaactcagtgtagtgtgcgtaattcaatatcatact

SEQ ID NO: 107 - RNA, 45 nt, synthetic construct
  misc_feature 1..45: note = synthetic
  source 1..45: mol_type = other RNA
gggactacctcacactcgaggtagtgataagtaagatttggaagt

SEQ ID NO: 108 - RNA, 45 nt, synthetic construct
  misc_feature 1..45: note = synthetic
  source 1..45: mol_type = other RNA
gggacataaacctatgagtttatgtgcgatatggagtatttgaaa

SEQ ID NO: 109 - RNA, 45 nt, synthetic construct; identical to SEQ ID NO: 99
  misc_feature 1..45: note = synthetic
  source 1..45: mol_type = other RNA

SEQ ID NO: 110 - RNA, 45 nt, synthetic construct
  misc_feature 1..45: note = synthetic
  source 1..45: mol_type = other RNA
gggacgaagcataaagttgcttcgtactatctatcttatactaac

SEQ ID NO: 111 - RNA, 45 nt, synthetic construct; identical to SEQ ID NO: 97
  misc_feature 1..45: note = synthetic
  source 1..45: mol_type = other RNA

SEQ ID NO: 112 - RNA, 45 nt, synthetic construct; identical to SEQ ID NO: 100
  misc_feature 1..45: note = synthetic
  source 1..45: mol_type = other RNA

SEQ ID NO: 113 - RNA, 45 nt, synthetic construct
  misc_feature 1..45: note = synthetic
  source 1..45: mol_type = other RNA
gggacatactaacaatctagtatgtgaatgatggatactttgaaa

SEQ ID NO: 114 - RNA, 45 nt, synthetic construct
  misc_feature 1..45: note = synthetic
  source 1..45: mol_type = other RNA
gggcgaacgacgaaacggtcgttcggaatctactaaatcttgaaa

SEQ ID NO: 115 - RNA, 45 nt, synthetic construct
  misc_feature 1..45: note = synthetic
  source 1..45: mol_type = other RNA
gggcacataacataacggttatgtgacggatagtactaattgaaa

SEQ ID NO: 116 - RNA, 45 nt, synthetic construct
  misc_feature 1..45: note = synthetic
  source 1..45: mol_type = other RNA
gggcacctcacctacgtgtgaggtggcattgtatcatcttcgtat

SEQ ID NO: 117 - RNA, 45 nt, synthetic construct
  misc_feature 1..45: note = synthetic
  source 1..45: mol_type = other RNA
gggacactaatactacgattagtgtgataagtttgaaagtataag

SEQ ID NO: 118 - RNA, 45 nt, synthetic construct
  misc_feature 1..45: note = synthetic
  source 1..45: mol_type = other RNA
gggacctcacttacattagtgaggtgatggaatttgaaggtaatg

SEQ ID NO: 119 - RNA, 45 nt, synthetic construct; identical to SEQ ID NO: 95
  misc_feature 1..45: note = synthetic
  source 1..45: mol_type = other RNA

SEQ ID NO: 120 - RNA, 45 nt, synthetic construct
  misc_feature 1..45: note = synthetic
  source 1..45: mol_type = other RNA
gggacatagacaatttggtctatgtgaatcttgtaagtgaatatg

SEQ ID NO: 121 - RNA, 45 nt, synthetic construct
  misc_feature 1..45: note = synthetic
  source 1..45: mol_type = other RNA
gggcgaacaagaacatgcttgttcggaatctagtaatcacatcta

SEQ ID NO: 122 - RNA, 45 nt, synthetic construct
  misc_feature 1..45: note = synthetic
  source 1..45: mol_type = other RNA
gggaacctaatgtacgaattaggttgaattgaagtatatggaatc

SEQ ID NO: 123 - RNA, 45 nt, synthetic construct
  misc_feature 1..45: note = synthetic
  source 1..45: mol_type = other RNA
gggcaaatagatagagctctatttggataggattaggaattgaaa

SEQ ID NO: 124 - RNA, 45 nt, synthetic construct
  misc_feature 1..45: note = synthetic
  source 1..45: mol_type = other RNA
gggcaatagatcaaactatctattggcgtttatctgatttgtatg

SEQ ID NO: 125 - RNA, 45 nt, synthetic construct
  misc_feature 1..45: note = synthetic
  source 1..45: mol_type = other RNA
gggcacataaactaatctttatgtgtaatgataacttggattgaa

SEQ ID NO: 126 - RNA, 45 nt, synthetic construct
  misc_feature 1..45: note = synthetic
  source 1..45: mol_type = other RNA
gggcaagaataacattctattcttgtaattcgtttcgatcatatg

SEQ ID NO: 127 - RNA, 45 nt, synthetic construct
  misc_feature 1..45: note = synthetic
  source 1..45: mol_type = other RNA
gggcgaataagaatgcgcttattcggaatcactatcttcattaac

SEQ ID NO: 128 - RNA, 45 nt, synthetic construct
  misc_feature 1..45: note = synthetic
  source 1..45: mol_type = other RNA
gggaacatacaaattgatgtatgttgatagacacggtgagataga

SEQ ID NO: 129 - RNA, 45 nt, synthetic construct
  misc_feature 1..45: note = synthetic
  source 1..45: mol_type = other RNA
gggcatatagaatagagtctatatgtaatcgtcaattggagtaga

SEQ ID NO: 130 - RNA, 45 nt, synthetic construct
  misc_feature 1..45: note = synthetic
  source 1..45: mol_type = other RNA
gggcaaatgatcatcgtatcatttggcgtattcacttcattaaca

SEQ ID NO: 131 - RNA, 45 nt, synthetic construct
  misc_feature 1..45: note = synthetic
  source 1..45: mol_type = other RNA
gggctacactaaacaggtagtgtaggcggtaagataatgggagta

SEQ ID NO: 132 - RNA, 45 nt, synthetic construct
  misc_feature 1..45: note = synthetic
  source 1..45: mol_type = other RNA
gggcaatagaaataccgttctattgcatctttactcatatcgatt

SEQ ID NO: 133 - RNA, 45 nt, synthetic construct
  misc_feature 1..45: note = synthetic
  source 1..45: mol_type = other RNA
gggcatacctctacaatgaggtatggaatagaatggagtagtaag

SEQ ID NO: 134 - RNA, 45 nt, synthetic construct
  misc_feature 1..45: note = synthetic
  source 1..45: mol_type = other RNA
gggactccacgaatatccgtggagtgatagtagtttaagtcgtag

SEQ ID NO: 135 - RNA, 45 nt, synthetic construct
  misc_feature 1..45: note = synthetic
  source 1..45: mol_type = other RNA
gggcgaaatacaataccgtatttcgcatctttaagttcacatcga

SEQ ID NO: 136 - RNA, 45 nt, synthetic construct
  misc_feature 1..45: note = synthetic
  source 1..45: mol_type = other RNA
gggctcaacataacatgatgttgagaatgatgaataggattgaga

SEQ ID NO: 137 - RNA, 45 nt, synthetic construct
  misc_feature 1..45: note = synthetic
  source 1..45: mol_type = other RNA
gggagcgaacaataacgtgttcgctgatgattctaattcttaaca

SEQ ID NO: 138 - RNA, 45 nt, synthetic construct
  misc_feature 1..45: note = synthetic
  source 1..45: mol_type = other RNA
gggactccaagataagccttggagtaaagattatcggattgaatg

SEQ ID NO: 139 - RNA, 45 nt, synthetic construct
  misc_feature 1..45: note = synthetic
  source 1..45: mol_type = other RNA
gggagcgaacgcgaactcgttcgctcaatctcatcattcattaag

SEQ ID NO: 140 - RNA, 45 nt, synthetic construct
  misc_feature 1..45: note = synthetic
  source 1..45: mol_type = other RNA
gggcgaagatagatacatatcttcgaatacttacttctaaatcta

SEQ ID NO: 141 - RNA, 45 nt, synthetic construct
  misc_feature 1..45: note = synthetic
  source 1..45: mol_type = other RNA
gggacatatactttcatgtatatgtaaagtatctaagtaaatgaa

SEQ ID NO: 142 - RNA, 45 nt, synthetic construct
  misc_feature 1..45: note = synthetic
  source 1..45: mol_type = other RNA
gggacggaataacactctattccgtcatttcatttcaattcgtaa

SEQ ID NO: 143 - RNA, 45 nt, synthetic construct
  misc_feature 1..45: note = synthetic
  source 1..45: mol_type = other RNA
gggcactccaataggatttggagtgtaatagtagggtaaatgaga

SEQ ID NO: 144 - RNA, 45 nt, synthetic construct
  misc_feature 1..45: note = synthetic
  source 1..45: mol_type = other RNA
gggactccaactaaaccgttggagtgatatcggtaagtaatagaa

SEQ ID NO: 145 - RNA, 45 nt, synthetic construct
  misc_feature 1..45: note = synthetic
  source 1..45: mol_type = other RNA
gggcacctaactaacatgttaggtgtaatagtgaagtatgagatg

SEQ ID NO: 146 - RNA, 45 nt, synthetic construct
  misc_feature 1..45: note = synthetic
  source 1..45: mol_type = other RNA
gggcattaagttaagttacttaatggatggatagatggagatgat

SEQ ID NO: 147 - RNA, 45 nt, synthetic construct
  misc_feature 1..45: note = synthetic
  source 1..45: mol_type = other RNA
gggacctaaactaacgcgtttaggtcaatcactcttaatctacta

SEQ ID NO: 148 - RNA, 45 nt, synthetic construct
  misc_feature 1..45: note = synthetic
  source 1..45: mol_type = other RNA
gggacttaaacgaaacggtttaagtgcacgtattggagattggga

SEQ ID NO: 149 - RNA, 62 nt, synthetic construct
  misc_feature 1..62: note = synthetic
  source 1..62: mol_type = other RNA
gggctgcaggtgacagatatcacctgcagcgataaccctaccctacccttgtatactttgat

SEQ ID NO: 150 - RNA, 62 nt, synthetic construct; identical to SEQ ID NO: 80
  misc_feature 1..62: note = synthetic
  source 1..62: mol_type = other RNA

SEQ ID NO: 151 - RNA, 62 nt, synthetic construct
  misc_feature 1..62: note = synthetic
  source 1..62: mol_type = other RNA
gggtgacatatccaagagcggatatgtcactaacatcaacatcaagcgttctatactttact

SEQ ID NO: 152 - RNA, 62 nt, synthetic construct; identical to SEQ ID NO: 79
  misc_feature 1..62: note = synthetic
  source 1..62: mol_type = other RNA

SEQ ID NO: 153 - RNA, 62 nt, synthetic construct
  misc_feature 1..62: note = synthetic
  source 1..62: mol_type = other RNA
gggacgtgcgcagctcctactgcgcacgtcaatcttcaatctatacatcatttagtcttagt

SEQ ID NO: 154 - RNA, 62 nt, synthetic construct
  misc_feature 1..62: note = synthetic
  source 1..62: mol_type = other RNA
gggtgacctgaaccgaactgttcaggtcaagaacaagataacagacctacataaactaagaa

SEQ ID NO: 155 - RNA, 62 nt, synthetic construct
  misc_feature 1..62: note = synthetic
  source 1..62: mol_type = other RNA
gggagtgccacaggctcgactgtggcactcaaatctaaactcataccgtaatcttaagtcgt

SEQ ID NO: 156 - RNA, 62 nt, synthetic construct
  misc_feature 1..62: note = synthetic
  source 1..62: mol_type = other RNA
gggaccctactccagcacaggagtagggtacaagcatcaaatcaatcaatacacatacctaa

SEQ ID NO: 157 - RNA, 62 nt, synthetic construct
  misc_feature 1..62: note = synthetic
  source 1..62: mol_type = other RNA
gggtcactactgggatataccagtagtgaagactaaagacaacgcactcaccttaccttact

SEQ ID NO: 158 - RNA, 62 nt, synthetic construct
  misc_feature 1..62: note = synthetic
  source 1..62: mol_type = other RNA
gggacgttagtcgatgtcgcgactaacgtagcagacaagacagacacatacacataaacaga

SEQ ID NO: 159 - RNA, 62 nt, synthetic construct
  misc_feature 1..62: note = synthetic
  source 1..62: mol_type = other RNA
gggtcggtgcgttaacggcaacgcaccgacttactaacatcaacgcattctatcattaagct

SEQ ID NO: 160 - RNA, 62 nt, synthetic construct; identical to SEQ ID NO: 151
  misc_feature 1..62: note = synthetic
  source 1..62: mol_type = other RNA

SEQ ID NO: 161 - RNA, 62 nt, synthetic construct
  misc_feature 1..62: note = synthetic
  source 1..62: mol_type = other RNA
gggacgcgctagcgcatcggctagcgcgtagatagaacaagacagaccatacacatacaact

SEQ ID NO: 162 - RNA, 62 nt, synthetic construct
  misc_feature 1..62: note = synthetic
  source 1..62: mol_type = other RNA
gggagccatgatcgcccaggatcatggctctaaatcaacataacagcatcaatctttatcat

SEQ ID NO: 163 - RNA, 62 nt, synthetic construct; identical to SEQ ID NO: 162
  misc_feature 1..62: note = synthetic
  source 1..62: mol_type = other RNA

SEQ ID NO: 164 - RNA, 62 nt, synthetic construct
  misc_feature 1..62: note = synthetic
  source 1..62: mol_type = other RNA
gggacaccggaggcgtgcacctccggtgtagaacgaacatacaacgacacctttactttact

SEQ ID NO: 165 - RNA, 62 nt, synthetic construct
  misc_feature 1..62: note = synthetic
  source 1..62: mol_type = other RNA
gggacgatatacgggtctacgtatatcgtacaagaaccatacaagacaacgacgacactaga

SEQ ID NO: 166 - RNA, 62 nt, synthetic construct
  misc_feature 1..62: note = synthetic
  source 1..62: mol_type = other RNA
gggcagggtacttgtactgaagtaccctgagcagagacaaatacagacaactacaatactaa

SEQ ID NO: 167 - RNA, 62 nt, synthetic construct; identical to SEQ ID NO: 149
  misc_feature 1..62: note = synthetic
  source 1..62: mol_type = other RNA

SEQ ID NO: 168 - RNA, 62 nt, synthetic construct
  misc_feature 1..62: note = synthetic
  source 1..62: mol_type = other RNA
gggccatacctggcaactaccaggtatggctaagaacctacagaaccgtataagtatccagt

SEQ ID NO: 169 - RNA, 62 nt, synthetic construct
  misc_feature 1..62: note = syntehtic
  source 1..62: mol_type = other RNA
gggagccgtgcagcgtgcactgcacggctagataagaacaagaacgacataccatacactaa

SEQ ID NO: 170 - RNA, 62 nt, synthetic construct
  misc_feature 1..62: note = sythetic
  source 1..62: mol_type = other RNA
gggttcccgtcactggtacgtgacgggaacaatacaagacaagacgaaatcaattagtacgt

SEQ ID NO: 171 - RNA, 62 nt, synthetic construct
  misc_feature 1..62: note = synthetic
  source 1..62: mol_type = other RNA
gggacctgctcaccttcatgtgagcaggtagacgaacaagaacaagacacatacaatacact

SEQ ID NO: 172 - RNA, 62 nt, synthetic construct
  misc_feature 1..62: note = synthetic
  source 1..62: mol_type = other RNA
gggaccgtaacgaaatccatcgttacggtaagataaacagaacaagacataaagacacagga

SEQ ID NO: 173 - RNA, 62 nt, synthetic construct
  misc_feature 1..62: note = synthetic
  source 1..62: mol_type = other RNA
gggacatcagagactagtatctctgatgtataagacaagacataagaacagaaatacacaga

SEQ ID NO: 174 - RNA, 62 nt, synthetic construct
  misc_feature 1..62: note = synthetic
  source 1..62: mol_type = other RNA
gggagtcacgcgctacgttgcgcgtgactagaacatacataaacagaccattacacatacat

SEQ ID NO: 175 - RNA, 62 nt, synthetic construct
  misc_feature 1..62: note = synthetic
  source 1..62: mol_type = other RNA
gggagtccttgaggtgcgactcaaggactcagaacaagacaaagacctatacgattaagatt

SEQ ID NO: 176 - RNA, 62 nt, synthetic construct
  misc_feature 1..62: note = synthetic
  source 1..62: mol_type = other RNA
gggccacttctgactgcgatcagaagtggcagaacaagatacaagacacatttactactact

SEQ ID NO: 177 - RNA, 62 nt, synthetic construct
  misc_feature 1..62: note = synthetic
  source 1..62: mol_type = other RNA
gggaccctcattgctctaccaatgagggtagcaacataaacataacctactttcaacttact